(19) United States
(12) Patent Application Publication
(10) Pub. No.: US 2026/0024454 A1
(43) Pub. Date: Jan. 22, 2026

(54) SYSTEMS AND METHODS FOR MENTAL AND BEHAVIORAL HEALTH CARE AND MANAGEMENT

(71) Applicant: Vital Start Health Inc., Philadelphia, PA (US)

(72) Inventors: Kirthika Parmeswaran, Philadelphia, PA (US); Johnny Chuo, Philadelphia (UA)

(73) Assignee: Vital Start Health Inc., Philadelphia, PA (US)

(21) Appl. No.: 18/872,045

(22) PCT Filed: Jun. 14, 2023
(86) PCT No.: PCT/US2023/068444

§ 371 Date: Dec. 5, 2024

Related U.S. Application Data
(60) Provisional application No. 63/366,328, filed on Jun. 14, 2022.

(51) Int. Cl. G09B 5/14; G16H 20/70; G16H 40/67

(52) U.S. Cl. CPC G09B 5/14; G16H 20/70; G16H 40/67

(57) ABSTRACT

Systems and methods for behavioral and mental healthcare management. A system is comprised of a web-based platform and a device configured for an experiential virtual therapy session, such as a virtual reality (VR) and/or an augmented reality (AR) device. The therapy session is experienced by the patient alone or is simultaneously experienced by the patient and a healthcare practitioner such that the practitioner can directly observe the patient's reactions within the context of the simulation and provide real-time feedback during the session to help the patient develop coping skills. The system implements novel analytical approaches for clinicians to quantify and manage multidimensional aspects of mental health to improve the health and well-being of the patient for measurable behavioral health.

### CROSS-REFERENCE(S) TO RELATED APPLICATION(S)

This application claims the benefit of U.S. provisional patent application 63/366,328, filed on Jun. 14, 2022, which is incorporated by reference herein in its entirety.

### BACKGROUND

Maladaptive responses to trauma can lead to the development or progression of mental health disorders. For women and childbirth, Perinatal Mood and Anxiety Disorders (PMADs) are the most common complication of childbirth with an economic impact upwards of $14 billion. Close to 15% of postpartum women in the U.S. suffer from PMADs annually, with 28-70% for mothers of infants hospitalized in a Neonatal Intensive Care Unit (NICU). In addition, according to the World Health Organization (WHO), 20% of mothers worldwide are similarly affected with PMADs. However, despite the need, over 50% of mothers with PMADs do not receive adequate treatment, and existing approaches for treatment are not effective. In addition, this population is at particular risk for Post-Traumatic Stress Disorder (PTSD) with prevalence as high as 88% of mothers of infants with congenital defects. Although evidence-based interventions exist to prevent and treat PTSD, no market solution currently exists to provide specialized care for at-risk PTSD mothers who are exposed to sights, sounds, and situations in the NICU that can provoke trauma responses. Further, care providers may be geographically inaccessible due to lack of access to transportation or a poor professional fit for treatment of PMAD, PTSD, and other conditions.

This problem is exacerbated with the rise in complications in pregnancy and childbirth, which is 21% in the U.S., according to a Blue Cross and Blue Shield Report. Those trying to conceive and those using reproductive treatments to become pregnant are also affected. There are 55,000 babies born each year in the U.S. through fertility treatments and 90% of the women going through it reported depression.

Accordingly, there is a need for improved approaches for telemedicine and virtual therapy for providing mental and behavioral health services, including for those trying to conceive, expectant mothers, and perinatal populations across pregnancy and postpartum. The present disclosure addresses this unmet need.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

The disclosure relates to systems and methods for managing behavioral and mental health. A system includes a platform for administering immersive therapy and mental health services to a patient and for facilitating care of the patient based on patient input or feedback. The platform implements novel approaches for quantifying and representing the health of patients to caretakers to facilitate decision-making and improve quality of care.

In general, the disclosure provides systems and methods for immersive therapeutic experiences for the prevention and treatment of mental health disorders, including in the context of reproductive mental health as well as other situations such as cancer diagnosis, treatment, and loss and grief. A system includes a web-based platform, accessible by patient devices and practitioner devices, that is configured to administer a therapy session (i.e., psychotherapy, coaching, counseling, psychoeducation, etc.) to a patient. The platform is configured to simultaneously administer the therapy session to the patient and a therapy management session to the practitioner, such that the practitioner can directly observe the patient in real-time during the therapy session and provide guidance or encouragement to the patient to help the patient manage emotions and develop mental fortitude.

The platform provides improved analytical tools to help clinicians evaluate patients and track their progress longitudinally in response to non-therapy events and therapy sessions. A video library is developed and used for a series of immersive therapy sessions for the patient, and the subjective experience of a particular video range from relaxational to psychoeducational to experiential. Relaxational sessions are used to help patients manage ongoing symptoms of stress, psychoeducational sessions are used to provide the practitioner the opportunity to educate the patient (e.g., CBT, others), and immersive sessions are used to reinforce healthy coping mechanisms and help the patient develop mental fortitude. An object of the disclosure is to provide enhanced systems and methods for improved telemedicine and virtual therapy that allows patients to experience immersive therapy and to face mental challenges in a controlled and supportive environment. This approach is called stress inoculation, and in embodiments, the disclosed platform implements this approach for reproductive and maternity mental health, paternity mental health, and wellness/well-being.

Another object of the present disclosure is to provide systems and platforms that are more readily accessible to individuals who need mental and behavioral health services, including the general population as well as the underserved, women, minorities, and economically disadvantaged individuals.

In an aspect, the disclosure provides a system for mental healthcare management, the system comprising circuitry configured to simultaneously administer a virtual therapy session to a patient device and a virtual therapy management session to a practitioner device. The virtual therapy session and the virtual therapy management session are operably connected for exchange of data therebetween for an experience of virtual therapy by a patient and management of the experience by the patient, a practitioner, or both.

In another aspect, the disclosure provides a patient device configured for virtual therapy, the patient device comprising circuitry configured to access a virtual therapy session of a system for mental healthcare management and circuitry configured to depict media and practitioner input of the virtual therapy session to a patient. The patient device can be implemented as any of various devices; in embodiments, the patient device is a smartphone implemented as a virtual reality (VR) device or, alternatively, is a dedicated VR device.

In another aspect, the disclosure provides a practitioner device configured for virtual therapy management, the practitioner device comprising circuitry configured to access a virtual therapy management session of a system for mental healthcare management and circuitry configured to depict media and patient input of the virtual therapy management session to a practitioner.

In another aspect, the disclosure provides a method for mental healthcare management, the method comprising: depicting, to a patient and a practitioner, media for virtual therapy for the patient; receiving, from the patient, patient input related to an experience of the virtual therapy by the patient; and providing, by the practitioner, practitioner input related to the experience of the virtual therapy by the patient. The method can be implemented with one or more systems and/or devices of the disclosure.

Other objects, features and advantages of the present disclosure will become apparent from the following detailed description taken in conjunction with the accompanying drawings.

### DESCRIPTION OF THE DRAWINGS

The foregoing aspects and many of the attendant advantages of this disclosure will become more readily appreciated as the same become better understood by reference to the following detailed description, when taken in conjunction with the accompanying drawings, wherein:

FIG. 1 shows a flow chart providing a high-level overview of an example process for identifying and treating patients with virtual therapy, according to the disclosure.

FIG. 2A shows a higher-level diagram of an example system, according to the disclosure.

FIG. 2B shows a lower-level diagram of an example system, according to the disclosure.

FIG. 3A shows an example use of a first patient device in a method of virtual therapy.

FIG. 3B shows an example use of a second patient device in a method of virtual therapy.

FIG. 4A shows an example relaxational virtual therapy.

FIG. 4B shows an example experiential or stressful virtual therapy.

FIG. 4C shows an example experiential virtual therapy experienced by a patient and a practitioner.

FIG. 5A shows an example dashboard for the practitioner at a first view.

FIG. 5B shows the example dashboard for the practitioner at a second view.

FIG. 6A shows an example of a multi-dimensional axis for visual representation of the patient's health status.

FIG. 6B shows the multi-dimensional axis populated with normalized values of healthcare parameters of the patient.

FIG. 6C shows an example health status or wellness score of the patient based on the normalized values of healthcare parameters of the patient.

FIG. 7 shows an example computational device, e.g., a patient device, a system, and/or a practitioner device, according to the disclosure.

FIG. 8 shows an example use of the disclosure for treatment of a patient across multiple stages of the birthing process, including pre-conception, birthing, and postpartum.

FIG. 9 shows an example overview of stress inoculation therapy including anticipatory guidance, loss and grief, identity challenges, stress reduction, and sleep.

FIG. 10A shows a collection of media VR videos for anticipatory guidance, loss and grief, identity challenges, stress reduction, and sleep.

FIG. 10B shows an arrangement of media VR videos for anticipatory guidance, loss and grief, identity challenges, stress reduction, and/or sleep, arranged by the system for a first patient.

FIG. 10C shows an arrangement of media VR videos for anticipatory guidance, loss and grief, identity challenges, stress reduction, and/or sleep, arranged by the system for a second patient.

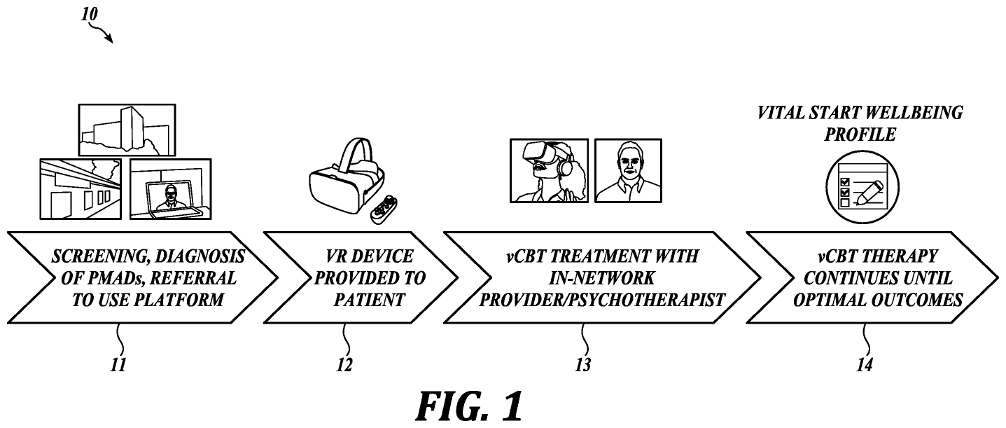

### DETAILED DESCRIPTION

Reference is made herein to the attached drawings. Like reference numerals may be used in the drawings to indicate like or similar elements of the description. The figures are intended for representative purposes, are not drawn to scale, and should not be considered limiting.

Unless otherwise defined herein, terms and phrases used in connection with the present disclosure shall have the meanings that are commonly understood by those of ordinary skill in the art.

Where a reference is made to a singular noun, whether with or without use of an indefinite or definite article (e.g., “a”, “an”, or “the”), this includes a plural of that noun unless something else is specifically stated. Furthermore, the terms first, second, third, and the like in the description and in the claims, are used for distinguishing between elements and not necessarily for describing a sequential or chronological order. It is to be understood that the terms so used are interchangeable under appropriate circumstances and that the implementations of the disclosure described herein are capable of operation in other sequences than described or illustrated herein.

As used herein, the term “about” refers to the usual error range for the respective value readily known to the skilled person in this technical field. Reference to “about” a value or parameter herein includes and describes implementations and embodiments that are directed to that value or parameter per se.

As used herein, the terms “therapy,” “therapy session,” and “immersive therapy session,” “coaching session,” “counseling session,” and the like, refer to any interaction between a person (e.g., a patient) and a system and/or a device of the present disclosure for the purpose of maintaining or improving mental or behavioral health of the person, and includes but is not limited to psychotherapy, coaching, counseling, group therapy, psychoeducation, and the like.

As used herein, terms that indicate an ability of an element to have a property or characteristic as part of a state of the element include states of the element in which it has the property or characteristic (e.g., is attachable and is attached) as well as states of the element in which it does not have the property or characteristic (e.g., is attachable but is not attached), unless something else is specifically stated. If an element is described as having a property or characteristic (e.g., is attached), this includes elements that conditionally have the property or characteristic (e.g., can be conditionally attached) as well as elements that unconditionally have the property or characteristic (e.g., can be unconditionally attached), unless something else is specifically stated.

### I. SYSTEMS AND DEVICES

The present disclosure provides systems that can be configured for telemedicine and immersive and experiential virtual therapy sessions. While shown and described in terms of telemedicine, remote care, and other situations in which a practitioner is remote to a patient, the systems and devices can also be used in situations in which the practitioner is local to the patient, e.g., within the same room or building (e.g., face to face), without departing from the scope of the present disclosure. In this manner, elements of the disclosure can be adapted for use according to need.

a. Systems and Virtual Reality and Augmented Reality (Vr/Ar) Devices

In embodiments a device, such as a smartphone, a tablet, a personal computer, a dedicated virtual reality/augmented reality (VR/AR) device, or a smartphone VR/AR device, is used to access one or more servers of a system for administration of care and immersive virtual therapy sessions. The device can be particular to the individual accessing the system; for example, a patient can access the server using a personal dedicated VR device or a personal smartphone VR device, and a practitioner can access the server using a workstation computer, for example.

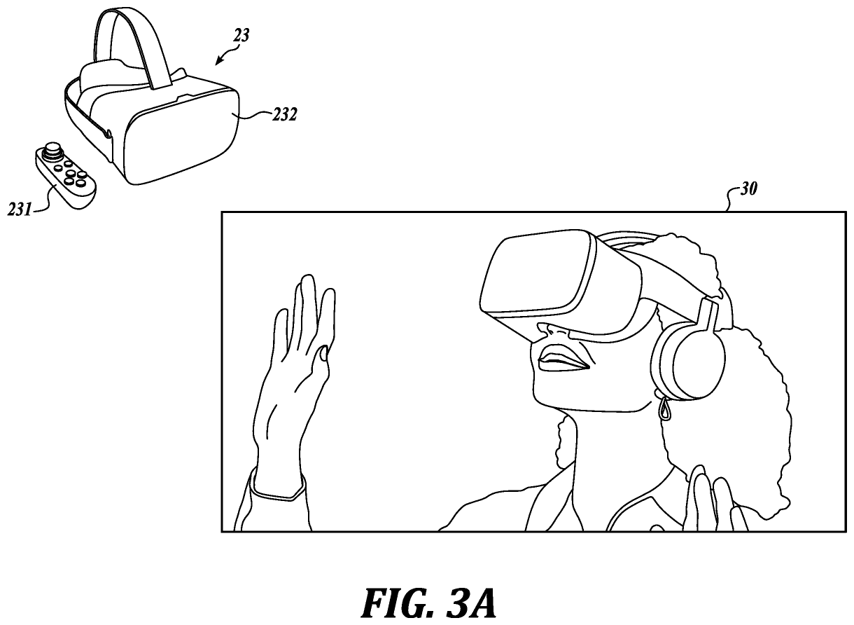

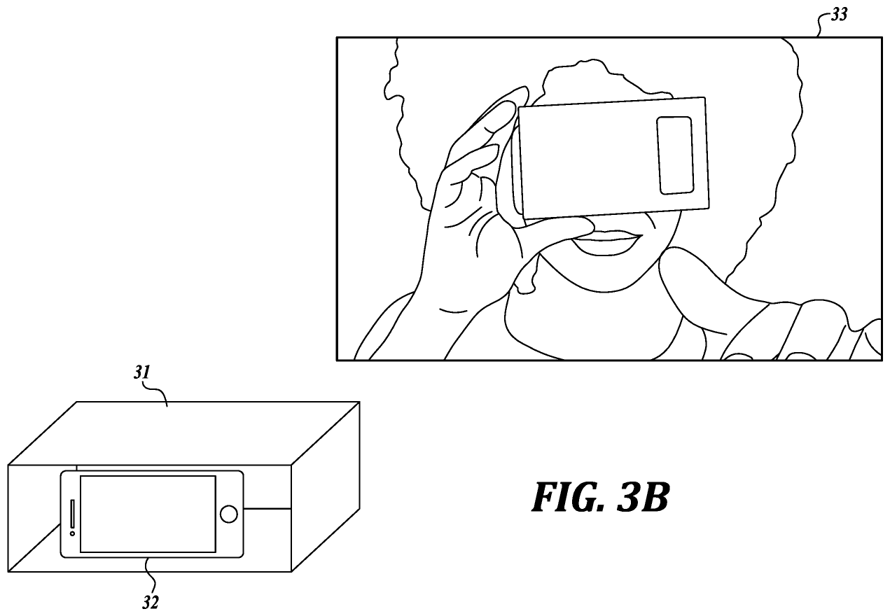

In embodiments, a dedicated VR/AR device (e.g., 23, 231, 232 of FIG. 3A) includes any device that is generally configured for a virtual reality and/or augmented reality experience 30, as shown at FIG. 3A. In embodiments, a smartphone VR/AR device (e.g., 31, 32 of FIG. 3B) is a low-cost alternative to a dedicated VR/AR device and includes a smartphone 32 or other personal computing device with an after-market addition 31 to enhance the immersive characteristics of the device when used for a therapy session 33, and in embodiments includes a cardboard or paper cutout 31 placed around the smartphone 32 to prevent ambient light from entering the patient's field of vision when viewing the device during the virtual therapy session 33, as shown at FIG. 3B.

A server includes any networked computer or computer system that is configured to receive requests, process requests, and transmit responses to client devices. In embodiments, one or more servers of a system are used to administer the virtual therapy session to a patient device and the virtual therapy management session to a practitioner device.

In general, a system is configured to simultaneously administer a virtual therapy session to one or more patients via one or more patient devices, optionally in combination with a virtual therapy management session to one or more practitioners via one or more practitioner devices. Because the therapy session is administered simultaneously with the therapy management session, practitioners are able to view the therapy session and directly observe patients during therapy sessions to provide real-time input and guidance for development of the patient's coping skills. A practitioner views a patient (or an anonymized avatar or virtual representation of the patient) through a live video feed, e.g., via a webcam, and communicates with the patient using video chat or web-based video conference. In the case of group therapy sessions with two or more patients, the patients similarly view each other (or an anonymized avatar or virtual representation) through a live video feed and communicate with each other using video chat or web-based video conferences.

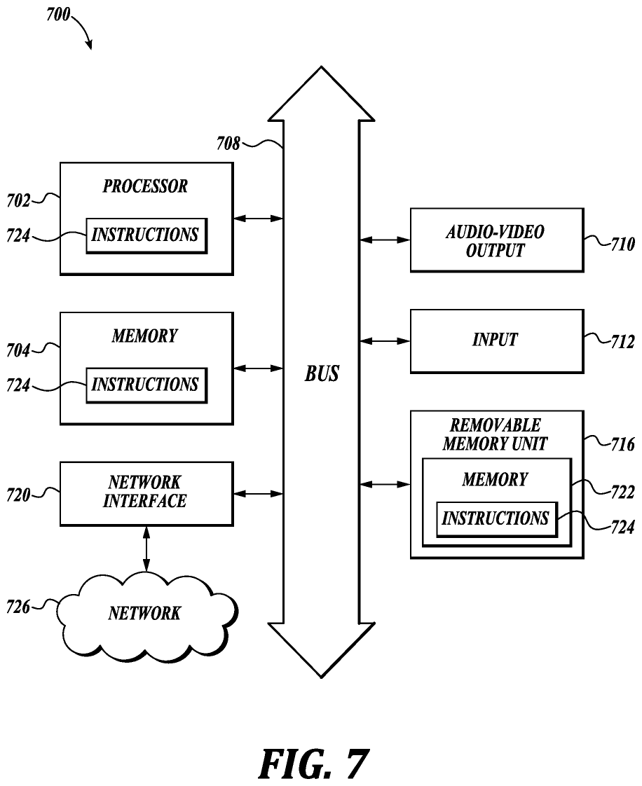

Referring now to FIG. 7, there is shown a diagram of components of an example device of the present disclosure. The device is shown in the example form of a computer device 700 with which instructions 724 can be executed to cause the device to perform any one or more of the operations and/or methodologies of the disclosure, in whole or in part, individually or in combination, in any order of events or steps. In embodiments, the device operates as a standalone device, or can be connected (e.g., networked) to other devices. In a networked deployment, the device can operate in the capacity of a server or a client device in a server-client network environment, or as a peer device in a peer-to-peer (or distributed) network environment. The device can be a server, a smartphone or cellular telephone, a tablet, a personal computer (PC), a dedicated virtual reality/augmented reality (VR/AR) device, a smartphone VR/AR device, a set-top box (STB), a personal digital assistant (PDA), a web appliance, a network router, switch, or bridge, or any device capable of executing instructions (sequential or otherwise) that specify actions to be taken by that device. Further, while only a single device is illustrated, the term “device” shall also be taken to include any collection of devices that individually or jointly execute a set (or multiple sets) of instructions to perform any one or more of the methodologies discussed herein.

The device 700 generally includes circuitry, such as dedicated hardware circuitry and/or processor circuitry, that configures the device 700 according to the disclosure. In the shown embodiment, the device 700 includes a processor 702 (e.g., a central processing unit (CPU), a graphics processing unit (GPU), or both), and a main memory 704, and can include a static memory, which communicate with each other via a bus 708. The device 700 can further include an audio-video output 710 (e.g., a video display, a liquid crystal display (LCD), or a cathode ray tube (CRT)). The device 700 also includes an input device 712 (e.g., a keyboard or a touch-sensitive display screen) and/or a user interface (UI) navigation (or cursor control) device (e.g., a mouse), a removable memory unit 716 (e.g., a disk drive unit), a signal generation device (e.g., a speaker), and a network interface device 720.

The memory 722 includes a non-transitory machine-readable medium (e.g., non-transitory computer memory) on which are stored one or more sets of data structures and instructions 724 (e.g., software) embodying or utilized by any one or more of the methodologies or functions described herein. The instructions 724 can also reside within the processor 702 and/or within memory 704, e.g., during execution thereof by the device 700, with the memory 704 and the processor 702 also constituting non-transitory machine-readable media.

While the machine-readable medium 722 is shown in an example embodiment to be a single medium, the term “machine-readable medium” can include a single medium or multiple media (e.g., a centralized or distributed database, and/or associated caches and servers) that store the one or more instructions 724 or data structures. The term “machine-readable medium” shall also be taken to include any tangible medium that is capable of storing, encoding, or carrying instructions 724 for execution by the device and that cause the device to perform any one or more of the methodologies of the present disclosure, or that is capable of storing, encoding, or carrying data structures utilized by or associated with such instructions 724. The term “machine-readable medium” shall accordingly be taken to include, but not be limited to, solid-state memories, and optical and magnetic media. Specific examples of machine-readable media 722 include non-volatile memory, including by way of example semiconductor memory devices, e.g., erasable programmable read-only memory (EPROM), electrically erasable programmable read-only memory (EEPROM), and flash memory devices; magnetic disks such as internal hard disks and removable disks; magneto-optical disks; and CD-ROM and DVD-ROM disks.

The instructions 724 can be transmitted or received over a communication network 726 using a transmission medium. The instructions 724 can be transmitted using the network interface device 720 and any one of a number of well-known transfer protocols (e.g., HTTP). Examples of communication networks include a local area network (LAN), a wide area network (WAN), the Internet, mobile telephone networks, plain old telephone (POTS) networks, and wireless data networks (e.g., Wi-Fi® and WiMax® networks). The term “transmission medium” shall be taken to include any intangible medium that is capable of storing, encoding, or carrying instructions 724 for execution by the machine, and includes digital or analog communications signals or other intangible media to facilitate communication of such software.

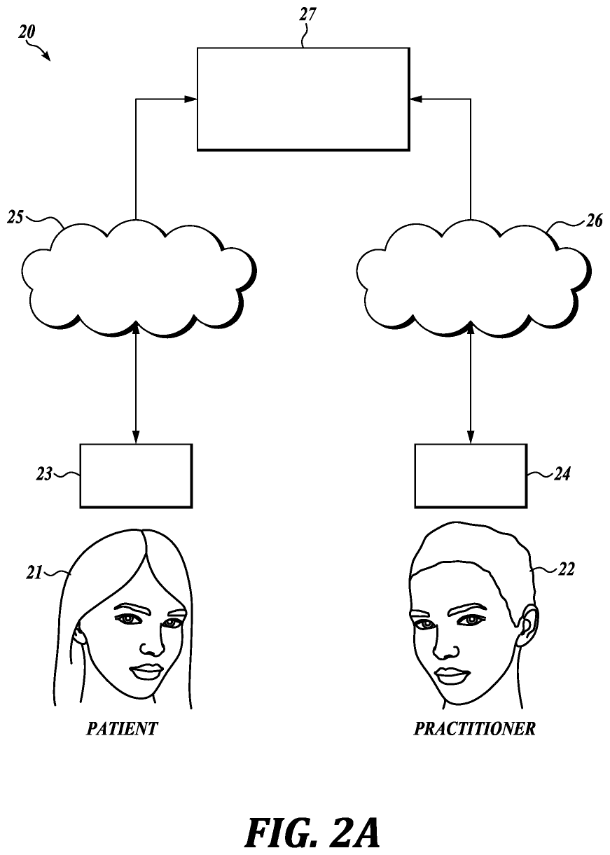

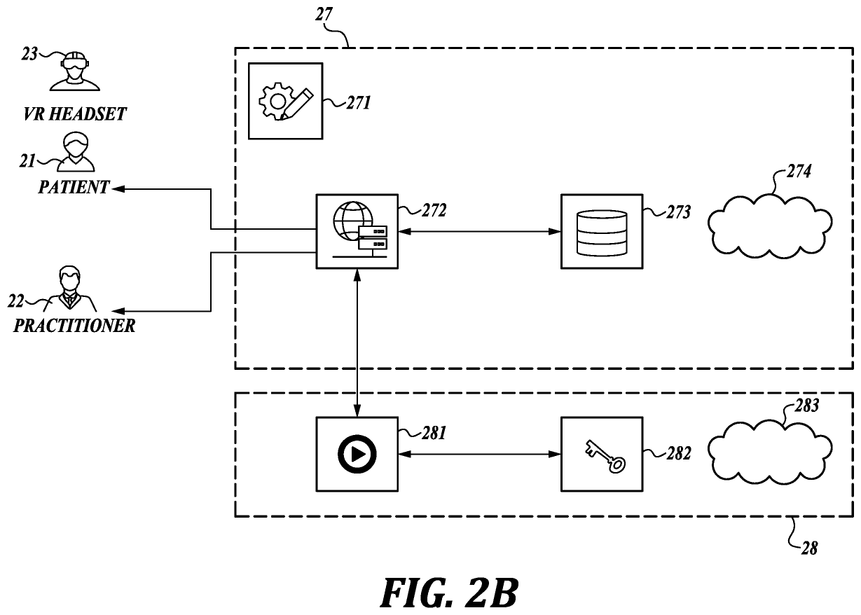

Referring now to FIG. 2A, there is shown a high-level overview of an example system 20. In various aspects, a system 20 for mental healthcare management for a patient 21 in need thereof comprises: circuitry 27 configured to simultaneously administer: a virtual therapy session to a patient device 23; and a virtual therapy management session to a practitioner device 24. The virtual therapy session and the virtual therapy management session are operably connected to each other for exchange of data therebetween for an experience of virtual therapy by a patient 21 and management of the experience by the patient 21, a practitioner 22, or both patient 21 and practitioner 22. In the shown embodiment, the patient device 23 is used to access a server 27 of the system 20 via one or more network connections 25, and the practitioner device 24 is used to access the server 27 of the system 20 via one or more network connections 26, for simultaneous administration of virtual therapy to the patient and virtual therapy management to the practitioner.

Referring now to FIG. 2B, there is shown a more detailed overview of the example system. In the shown embodiment, the patient 21 uses a VR headset 23 to access the server 27, and the practitioner 22 uses a workstation (not shown) to access the server 27. Server 27 includes control panel elements for customization 271, session elements for co-administering the virtual therapy session and the virtual therapy management session 272, and data storage elements 273 for storing data, such as patient and practitioner data, for later retrieval. Server 27 is shown as deployed as part of a cloud environment 274 that comprises a plurality of servers 27, for example.

In the shown embodiment, the elements for the virtual therapy session and the virtual therapy management session 272, or another element of the server 27, are operably connected to a media server 28 that stores and provides access to media 281, optionally with authentication elements 282. In the shown embodiment the media server 28 is deployed as part of a cloud environment 283 that can comprise a plurality of servers, for example. Media 281 includes audio media, video media, audiovisual media, and the like, that provides subject matter for use in virtual therapy.

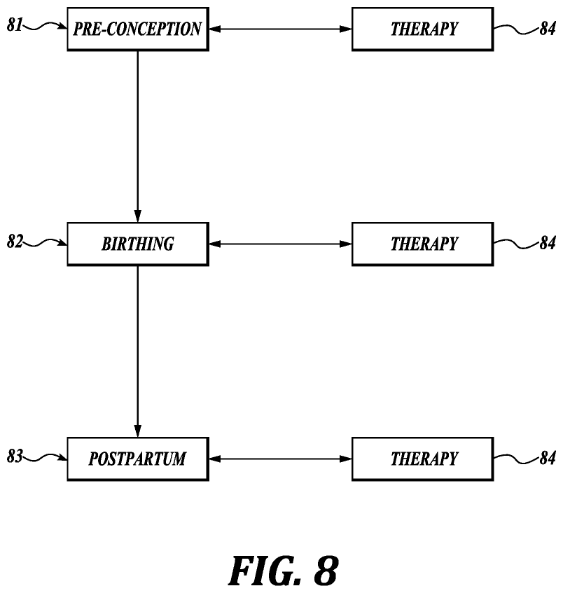

While the systems, devices, and methods of the disclosure are useful for any of a variety of mental health conditions and situations, examples disclosed herein relate to use for treatment of a patient across multiple stages of the birthing process, including pre-conception 81, birthing 82, and postpartum 83, as shown at FIG. 8. At each of these stages of the birthing process, one or more systems, devices, and/or methods can be used for coaching/therapy 84, as shown. In addition, the patient can experience different challenges or stressors at each stage 81, 82, 83, and the disclosure provides approaches that provide adapted coaching/therapy 84 to help the patient at each stage 81, 82, 83, since the patient's needs at each stage may be the same or different.

As a non-limiting example, as shown at FIG. 2B, if a patient needs preparation for a real-life NICU experience, media 281 comprises experiential videos of (real or staged) NICU experiences. Viewing the videos with the patient device, which provides an immersive experience of the NICU environment, can be stressful for the patient, who may be an expectant mother or father. The practitioner assists the patient with developing coping strategies during the therapy session to help with the real-life NICU experience (i.e., before, during, or after the real-life NICU experience), and this can increase robustness in the patient, lower the negative impact of the real-life NICU experience on the patient, and result in improved clinical outcomes for the patient. Similarly, in embodiments, media comprising videos of the birthing process or high-risk pregnancy including complications, or other situations, such as pricking a finger for gestational diabetes testing, is shown to the expectant mother and/or father. Also, similarly, in embodiments, media comprising videos of the reproductive journey, such as In-Vitro Fertilization (IVF) or Assisted Reproductive Technology (ART) can be shown to those trying to conceive to prepare them for the journey with psychoeducation and mental health support.

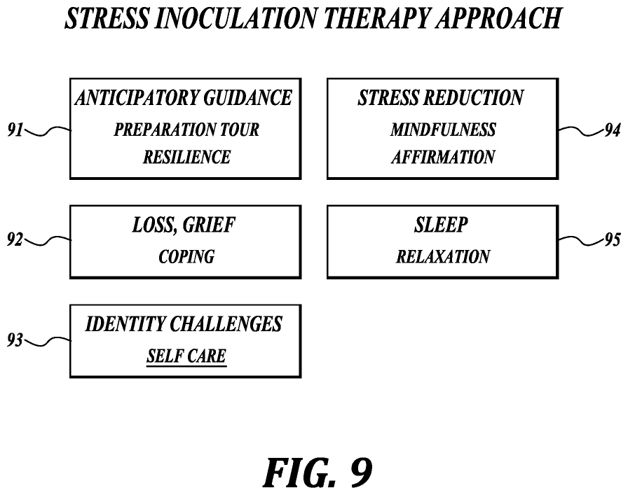

Accordingly, in embodiments, the systems, devices, and methods of the disclosure are configured for implementing stress inoculation therapy, as shown at FIG. 9. Stress inoculation therapy can include anticipatory guidance 91 (e.g., virtual preparation tours, resilience training, etc.), help with loss and grief 92 (e.g., coping skills), help with identity challenges 93 (e.g., self-care), help with stress reduction 94 (e.g., mindfulness, affirmation), and/or help with sleep 95 (e.g., relaxation techniques).

b. Patient Portal

In embodiments, the system is administered in whole or in part by one or more networked servers and includes a patient portal for administering access and therapy sessions to the patient device. In embodiments, the patient portal is comprised of a login prompt, in which case after logging into the system, the patient device gains access to a portal containing navigation features to assist the patient with accessing and experiencing an immersive therapy session. The patient device receives data and shows the data in an organized manner to facilitate navigation by the patient.

In embodiments, the patient portal provides access to a library of media (e.g., 281 of FIG. 2B) for immersive therapy sessions, or a subset of the library, e.g., as assigned by a mental healthcare practitioner or caretaker. The patient portal is used to browse, play, and pause AR/VR videos through a responsive web application. In embodiments, other functionalities are provided by the patient portal, such as the ability to view an appointment schedule (e.g., for psychotherapy, coaching, counseling, etc.), and the ability to initiate a virtual therapy session with a practitioner in real-time.

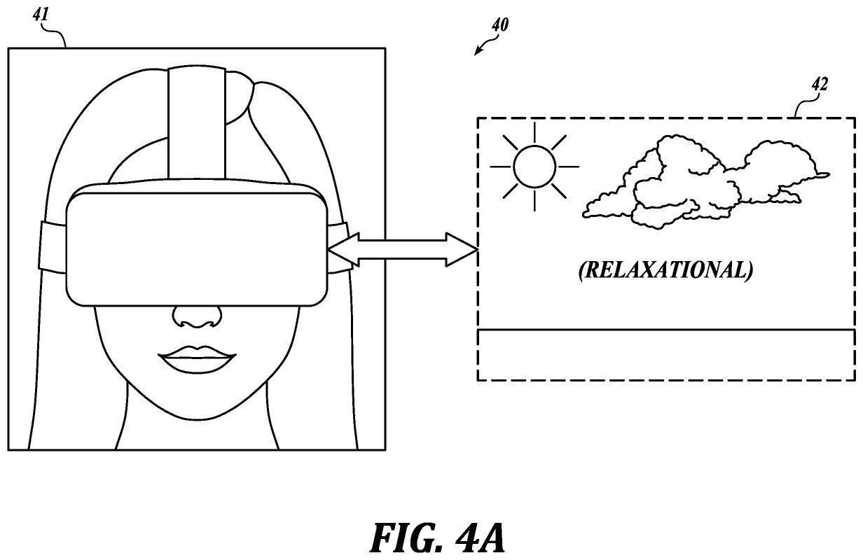

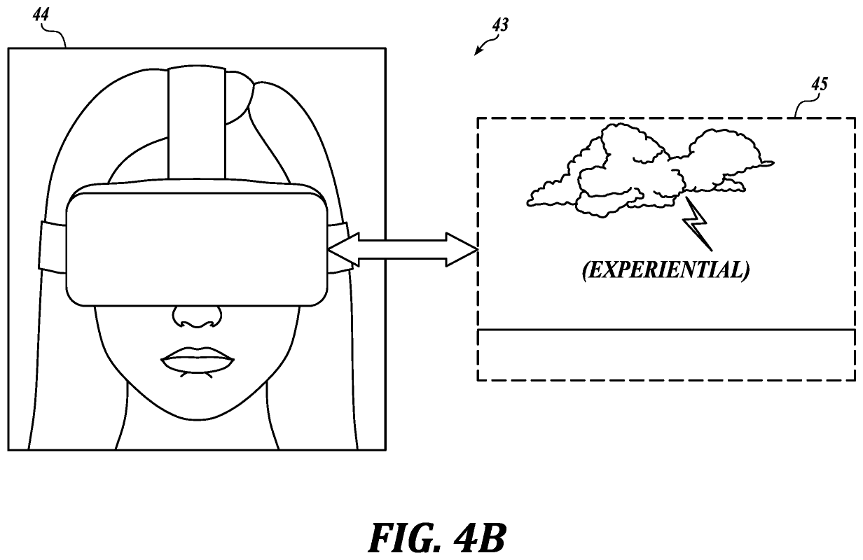

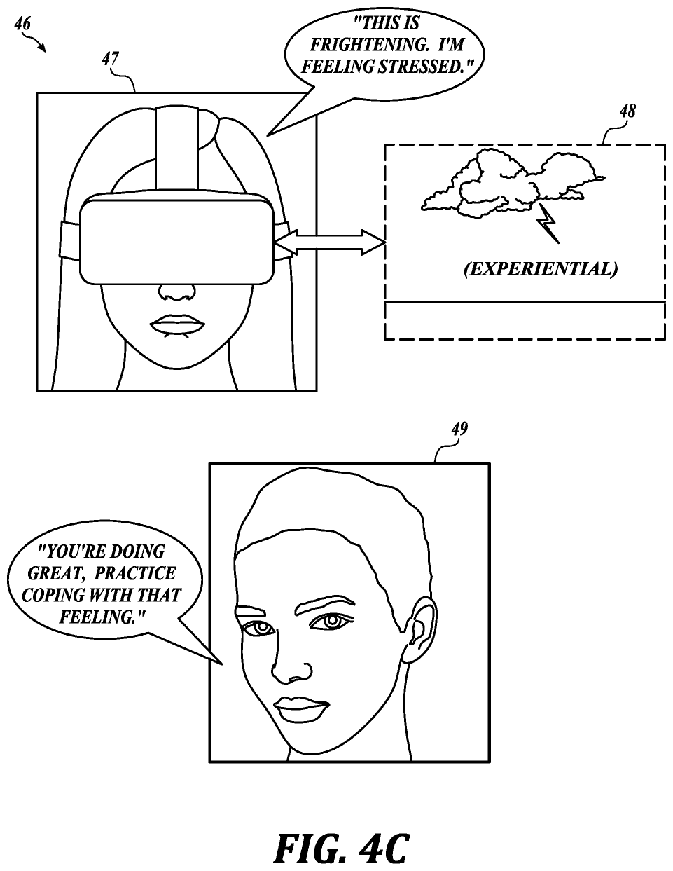

Referring now to FIGS. 4A, 4B, and 4C, there are shown an example relaxational virtual therapy 40 (FIG. 4A), an example experiential or stressful virtual therapy 43 (FIG. 4B), and an example experiential or stressful virtual therapy experienced by a patient and a practitioner 46 (FIG. 4C). In the shown embodiments, a patient wears a VR/AR device for the virtual therapy session, such that the patient experiences relaxational media 42 (FIG. 4A) or experiential media 45 (FIG. 4B). In embodiments, a practitioner 49 is able to view the patient 47, e.g., through a live webcam video stream, such that the practitioner 49 can observe responses, expressions, and statements or questions of patient 47 during the session 46 (FIG. 4C). In the shown embodiments, the patient 47 is experiencing stressful media 48 during the session and vocalizes a need for support or emotional reinforcement from the practitioner 49, who responds in turn with the requested support or guidance. The practitioner 49 is able to view both the patient 47 and the media 48 via a practitioner portal of the system.

c. Practitioner Portal

In embodiments, the system includes a practitioner portal for access and viewing by a healthcare practitioner, such as a psychologist, psychiatrist, nurse, doctor, therapist, etc. The practitioner portal can require a login from the practitioner before access is granted. The practitioner portal provides a virtual therapy management session and other administrative functionalities such as the ability to browse the AR/VR video library and assign content to patients, e.g., based on a past responsiveness of the patient or another patient the same or similar videos, or based on a past responsiveness of similar patients to the same or similar videos, etc., as may occur based on the practitioner's judgement and/or a recommendation engine of the system (e.g., an artificial intelligence and/or machine learning recommendation engine, or AI/ML engine). The practitioner portal provides access to a patient profile that displays information about the patient's progress and status and provides helpful information or suggestions for improving quality of care. This portal allows for different assessments, or metrics or surveys to be incorporated without code change for on-demand changes to the care protocol in the system.

i. Patient Profile

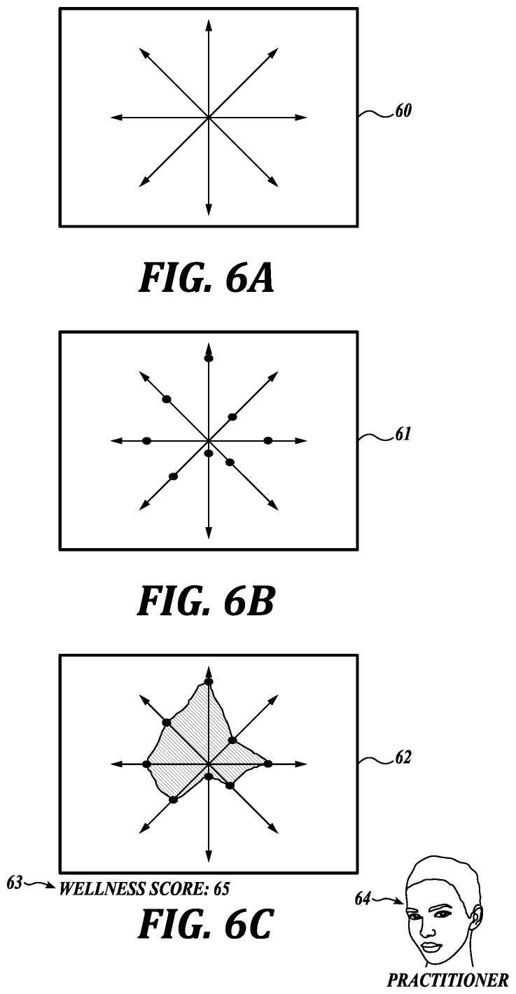

Referring now to FIGS. 6A, 6B, and 6C, there are shown a diagram as viewable by a practitioner through a practitioner portal of the system (FIG. 6A), the diagram with a set of normalized data points for a patient added (FIG. 6B), and the diagram with an area within the set of normalized data points highlighted, resulting in a wellness score or health status, in the form of a numerical score, for the patient (FIG. 6C). In embodiments, a diagram as shown at 60 of FIG. 6A is a component of a patient profile and includes a plurality of axes that each corresponds to a variable or healthcare parameter as quantitated by a patient input (e.g., a result from a survey or questionnaire that relate to physiological, behavioral, mental, or psychological metrics or psychometrics). Because different surveys for different healthcare parameters have different numerical rating scales, the data resulting from different surveys is normalized and shown on the patient profile using a normalized scale for comparison, as shown at 61 of FIG. 6B. In this manner, additional healthcare parameters using any particular scale can be normalized and included in the patient profile of the present disclosure.

In the shown example, each axis relates to a quantitative patient input related to quality of healthcare parameters such as anxiety, fatigue, sleep, stress, etc.; the closer the data point on an axis is to the middle intersection point, the lower the score for that parameter, and the further the data point on the axis is from the middle intersection point, the higher the score for that parameter. In embodiments, the area in the middle of the normalized data points is summed or otherwise quantitated 62 to produce a wellness score 63 for health status, as shown at FIG. 6C, to allow the practitioner 64 to quickly grasp overall wellness of the patient when viewing the patient profile.

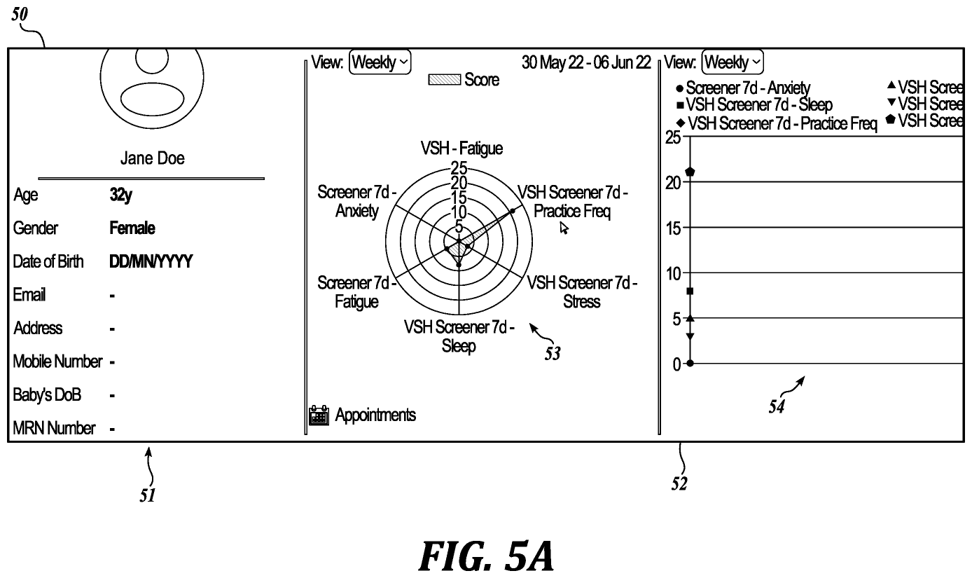

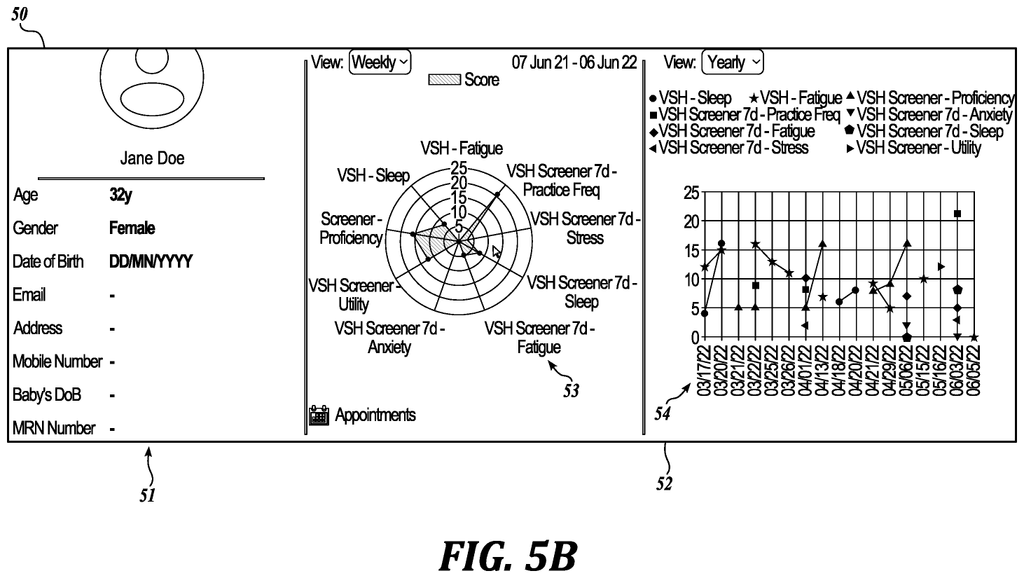

As shown at FIGS. 5A and 5B, a patient profile 50, with information about the patient 51, is depicted with data 52 from only one parameter or from a plurality of parameters 53, with data from only one snapshot in time or with data from a plurality of time points. In embodiments, data 52 is viewed as a graph 53 or chart 54 over time so the practitioner can better understand trends for the patient and more easily grasp responsiveness of the patient to particular therapies or treatments and make higher-quality recommendations or assign videos or immersive therapy modules to the patient based on data 52, which is in turn based on patient feedback and input. The use of patient input in response to clinical surveys and questionnaires in this manner helps ensure that clinical decisions are made with minimal bias and are informed based on the data 52.

ii. Coaching and Therapy Management

In embodiments, the practitioner portal provides access to tools and features for facilitating management of coaching and/or therapy for patients. In embodiments, these tools include therapy/coaching/counselling management tools for facilitating the creation and management (e.g., “mixing and matching”) of customized collections of immersive therapy 360-degree audio-video VR/AR assets (i.e., VR/AR asset playlists) for consumption by a particular patient (i.e., custom-made for one patient) or by a plurality of patients, such as patients having certain characteristics or features in common so as to be treatable with the same or similar treatment modalities. For example, if a group of patients all have a set of similar clinical features and are all favorably responsive to treatment modalities containing a majority of relaxational therapy sessions, then a software-implemented algorithm (e.g., an AI/ML algorithm configured to make recommendations; an AI/ML recommendation engine as an AI/ML model optionally a linear regression model) recommends a particular treatment modality comprised of mostly relaxational therapy sessions to patients in that group and future patients with the same or similar clinical features or characteristics. In at least some embodiments, the AI/ML playlist management feature makes recommendations alone and without substantial clinical oversight or, additionally or alternatively, makes suggestions to a clinician who then makes the clinical decision as to a treatment modality.

In embodiments, playlist management features are configured to seamlessly join or fade in/out videos within the playlist such that when the patient is immersed in a therapy session, he or she can view multiple videos without the jarring experience of videos abruptly stopping and starting. This improves the immersive “feeling” of the therapy session and encourages the patient to invest mentally and emotionally in the experience. This also helps reduce biases in the systems by having playlists developed with recommendations of patients in addition to practitioners.

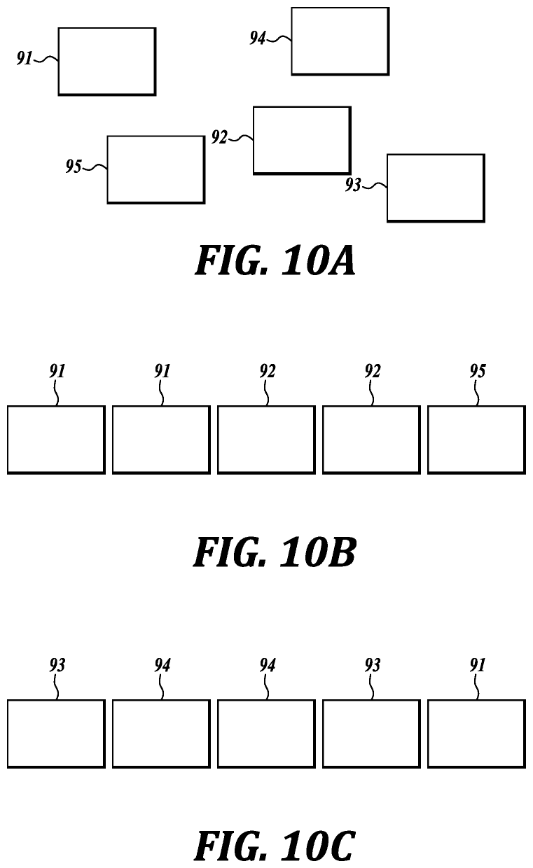

An example of “mixing and matching” media for particular patients is shown at FIGS. 10A, 10B, and 10C. As shown at FIG. 10A, a collection of media VR videos is provided for anticipatory guidance 91, loss and grief 92, identity challenges 93, stress reduction 94, and sleep 94. A first patient may respond better to, or may share features with one or more patients who respond better to, coaching/therapy related to anticipatory guidance 91 and loss and grief training 92; the system can therefore assign more of these trainings to the first patient, as shown at FIG. 10B. A second patient may respond better to, or may share features with one or more patients who respond better to coaching/therapy related to, identity challenges 93 and stress reduction 94; the system can therefore assign more of these trainings to the second patient, as shown at FIG. 10C. In this manner, the system “mixes and matches” or combines media configured for different types of stress inoculation therapy to produce customized therapy “playlists” effective for individual patients, as needed.

### II. METHODS

In embodiments, the systems, the devices, and the platforms administered by the systems are used in any of a variety of mental and behavioral health treatment and management methods. The methods generally involve administering a virtual therapy session to a device (e.g., a patient device), and in embodiments, includes quantifying a well-being of a patient (e.g., patient profile; wellness score) based on patient input (e.g., patient surveys or questionnaires), and managing a care plan of the patient based on the well-being (e.g., recommendation features, playlist features, and the like). In at least some embodiments, the methods are used by a patient by herself, by a patient with a practitioner present in the virtual therapy session for real-time practitioner input, or by a group of patients optionally with a practitioner present in the group virtual therapy session for facilitating the therapy session and providing real-time practitioner input to the group.

In embodiments, a method for mental healthcare management comprises depicting, to a patient and a practitioner, media for virtual therapy for the patient; receiving, from the patient, patient input related to an experience of the virtual therapy by the patient; and providing, by the practitioner, practitioner input related to the experience of the virtual therapy by the patient. In embodiments, the patient input related to the experience comprises a comment, question, remark, or other expression such as a gesture or facial expression or vocalization that indicates to the practitioner that their input is needed or would be helpful. In embodiments, the practitioner input is a response to the comment, question, remark, or other expression from the patient that provides guidance, education, reinforcement, or other helpful input to the patient during the immersive experience of the virtual therapy session.

a. Relaxational

As illustrated at FIG. 4A, a method for a relaxational virtual therapy session 40 comprises administering relaxational immersive VR/AR video media 42 to the patient 41 to help the patient relax or manage stress or anxiety. In embodiments, a treatment plan includes one or more relaxational therapy sessions positioned anywhere within a playlist for helping the patient relax. In embodiments, the relaxational therapy session is attended by the patient alone or in combination with other patients, and/or in combination with a practitioner. Example relaxational subject matter includes a video of a walk on a beach, a video of a meditation session, a video of a picnic at a park, etc. Any situation capable of being displayed with a VR/AR device that helps invoke feelings of relaxation in a patient can be used for a relaxational therapy session.

Relaxational subject matter can be generic or custom-made by or for a particular individual patient or a group of patients. Because subjective experience colors or defines what is relaxing for an individual, the exact subject matter implemented to invoke relaxation can vary between individuals. For example, one patient could find a VR/AR experience that involves a beach setting to be relaxational, but another may find this to be stressful due to a past negative experience at a beach. Relaxational therapy sessions can therefore be selected as a category by the practitioner, and a particular relaxational therapy session selected by the patient who knows what subject matter would help achieve a relaxational state.

b. Experiential

Another method of operation of the system includes an experiential mode that can be used for stress inoculation therapy, wherein the patient is exposed to stressors in a controlled environment to reinforce healthy coping with the stressors, as shown at FIGS. 4B and 4C. The experiential method of therapy includes immersing the patient within a virtual environment that is intentionally stressful to provide the patient with the opportunity to practice regulating thoughts, feelings, and emotions in a high-stress setting that is apart from real-world high-stress settings. In embodiments, the experiential methods of therapy are performed by the patient alone, by the patient as part of a group of patients, or in any of various combinations with a therapist or other practitioner who can provide support and guidance during the virtual therapy session.

In embodiments, experiential subject matter can be generic or customized to a particular individual or group of individuals. For example, if the system is to be used for patients with a common clinical background (e.g., high-risk parents with infants in intensive care units), then the experiential subject matter can take the form of subject matter that is relevant to that background (e.g., video of an infant with medical equipment connected to the infant, video of clinical staff working quickly or appearing stressed and using clinical terminology, etc.). Inasmuch as the subject matter helps invoke feelings of stress or anxiety or helps invoke other clinically-relevant thoughts or feelings in a particular patient, that subject matter can be used as experiential subject matter by that patient. In embodiments, the experiential subject matter is selected more specifically by the clinician or practitioner, who has professional insight as to what stressors the patient needs to work on coping with the most. In embodiments, the experiential therapy sessions are interspersed with relaxational therapy sessions, in any order or combination, as needed to help work toward clinical progress or to help improve day-to-day functioning of the patient.

### III. EXAMPLES

While the systems and methods are used for management of any of a variety of mental and behavioral health disorders, an example is provided to facilitate disclosure and understanding by the person skilled in the art.

a. Mitigation of Pmads in High-Risk Parents with Infants in Intensive Care Units

### Overview

In this example, the systems and methods of the disclosure are used for mitigation of perinatal mood and anxiety disorders (PMADs) as part of a novel application of an augmented and virtual reality (AR/VR) therapeutic tool, COURAGE. Specifically, the example examines COURAGE's feasibility and effectiveness in the preventive treatment of parents in the NICU at high risk for PTSD using principles of Cognitive Behavior Therapy (CBT) with personalized experiential practice.

COURAGE uses a novel approach, coined vCBT, that enables psychotherapists to directly observe how patients respond to exposure to stressors presented virtually during therapy sessions in order to intervene more appropriately using key validated measures. It was hypothesized that with this tool, and low-cost VR headsets, psychotherapists can help NICU parents reduce PTSD symptoms and thereby mitigate development of PTSD, a necessary step in demonstrating efficacy in this high-risk population.

The objective of the Phase I feasibility clinical randomized control study is to estimate vCBT's impact on trauma/PTSD, stress, depression, sleep, anxiety, and fatigue, which can estimate a priori values for a power calculation in a phase 2, clinical trial. The commercialization potential is significant; the addressable market for PMADs including PTSD is $16 billion; globally, 20% of mothers are impacted according to the World Health Organization (WHO). This innovative tool, delivering therapeutic experiential practices, paves a novel path not only to treat trauma experienced by parents of infants in the NICU, but also other psychological conditions.

The broader public health impact of this disclosure and example is to address the barriers to personalized, evidence-based psychotherapy treatment such as Cognitive Behavior Therapy (CBT) using an innovative digital therapeutic tool, COURAGE. The tool provides real-time therapist assistance (in person or remote, e.g., through virtual avatars) and personalized experiential practice through Virtual and Augmented Reality simulated experiences so patients can learn CBT skills and tools to successfully tackle real life stressors. Starting with the vital issue of mitigating Post-Traumatic Stress Disorder symptoms in parents of infants in the Neonatal Intensive Care Unit, COURAGE has the potential to disrupt the $201 Billion behavioral/mental health market and can be expanded to cater to other depressive and mental health disorders across adult and youth segments.

### Specific Aims

### Problem/Unmet Need

Perinatal Mood & Anxiety Disorders (PMADs) are the most common complication of childbirth with a significant economic impact. Close to 15% of postpartum women in the U.S. suffer from PMADs annually, with 28-70% for mothers of infants hospitalized in a Neonatal Intensive Care Unit (NICU). In addition, according to the World Health Organization (WHO), 20% of mothers worldwide are similarly affected with PMADs. These mothers are at risk for poor maternal-fetal attachment, which has been associated with impaired maternal-infant interaction and neurocognitive development in infants. Women of color are more prone to high-risk pregnancies and adverse perinatal outcomes.

Despite the need, over 50% of mothers with PMADs do not receive adequate treatment. These populations are at particular risk for Post-Traumatic Stress Disorder (PTSD) with prevalence as high as 88% of mothers of infants with congenital defects. Although evidence-based interventions to prevent and treat PTSD exist, no market solution currently exists to provide specialized care for mothers who are exposed to sights, sounds and situations in the NICU that can provoke trauma responses.

Solution/Technology: To mitigate this risk, it is proposed to apply and test a novel application of the virtual reality augmented with 3D model assets (AR/VR) therapeutic tool, COURAGE, of this disclosure. Specifically, it is aimed to examine COURAGE's feasibility and effectiveness in the preventive treatment of parents in the NICU at high risk for PTSD using principles of Cognitive Behavior Therapy (CBT) with personalized experiential practice.

COURAGE uses a novel approach that enables psychotherapists to directly observe how patients respond to exposure to stressors presented virtually during therapy sessions in order to intervene more appropriately. Using this approach, coined Virtual Cognitive Behavioral Therapy (vCBT), a psychotherapist can control the exposures and match the patients with AR/VR scenarios that are versions of real life visual and auditory stressors identified by the patient on a validated measure. With this tool, and low-cost VR headsets, psychotherapists can help NICU parents reduce PTSD symptoms and mitigate development of PTSD, a necessary step in demonstrating efficacy in high-risk populations.

An initial pilot study tested COURAGE with 10 parents of infants hospitalized at the NICU. A psychologist showed each parent 3 VR video exposures in a single session showcasing (a) NICU environment with alarm monitor, ventilator, and a nurse with a frail intubated mannequin baby, (b) Intubation process and (c) resuscitation procedure with neonatologist, respiratory specialist, nurse, and other staff in a tense environment. Before the session, psychometric measurements were taken including IES-R (Impact of Events Scale-Revised) for trauma/PTSD, after the session usability scores obtained followed by a repeat of the psychometric measures one week after the session.

There was a reduction in the IES-R measure (Mean −7.44) depicting improvement in trauma symptoms and that the outcomes were tending in a positive direction. Most parents did not seem to be having increased trauma symptoms from the VR exposures. In addition, there were also positive results observed using Likert scales to assess key critical use parameters, 1 to 5 for low to high, such as likelihood to use (Mean=4.14), ease of use (Mean=4.77), comfort level with VR equipment (Mean=4.22), and ability to talk with the therapist while using the system (Mean=4.55). When asked how the NICU VR experience made them feel, parents trended towards less stressful responses (Mean=2.66; min 1-max 3).

Given the promising research data, psychotherapy such as CBT, augmented with VR, enables targeted, more engaging, and more effective treatment.

### Specific Aims

Aim 1 of this feasibility study is to construct the set of relevant VR exposure scenarios (some superimposed with augmented reality (AR) 3D assets) to be viewed through an online COURAGE library viewer developed for this purpose. The new content is developed based on criteria from the Parental Stress Scale, NICU version (PSS:NICU). Parents of NICU infants (N=30) are asked to view and evaluate the stressfulness and relevance of the new content that is both matched and unmatched to their personal NICU experiences using the Subjective Units of Distress Scale (SUDS) in order to rate AR/VR scenario impact at inducing distress. Each parent is evaluated by a psychotherapist overseeing the session.

Aim 2 is a randomized controlled trial with 40 NICU parents having moderate to high PTSD symptoms (IES-R>=24). Parents are randomly assigned to either a vCBT group or CBT group and each receive 6 sessions (justified by prior CBT for PTSD research in the NICU6), 2 per week over 4 weeks starting at week 2. The vCBT uses relaxation training, guided imagery and exposure to NICU-specific AR/VR scenarios administered by psychotherapists using the COURAGE online viewer. The CBT group receives a scripted CBT program matching vCBT without video exposure. PTSD, depression, anxiety, maternal-infant attachment, fatigue, and sleep disturbances are evaluated, as well as attrition rate, usability, reliability, patient engagement, satisfaction, and overall perceived value. Results help estimate the power calculation for a future Phase 2, clinical trial.

### Research Strategy

### 1. Significance

Perinatal Mood and Anxiety Disorders (PMADs) are the most common complication of childbirth with an economic impact upwards of $14 billion. The CDC PRAMS report estimates that 15% of postpartum women suffer from PMADs annually, with rates significantly higher for mothers of infants hospitalized in a Neonatal Intensive Care Unit (NICU) (28-70%). This population is at particular risk for Post-Traumatic Stress Disorder (PTSD) with prevalence as high as 88% of mothers (66% of fathers) of infants with congenital defects relative to 5.6% for the general population of mothers following childbirth. Within the first 3-5 days of admission, 32% of parents experience onset of Acute Stress Disorder (ASD) which places them at risk for PTSD. In cases of infant death, 35% of mothers (30% of fathers) continue to have clinical PTSD thirteen months later. Moreover, PTSD is highly comorbid with depression as well as impaired maternal-infant bonding, attachment and interaction which all have been associated with impairments in infant growth and neurodevelopment. These problems in turn are associated with long term adverse biopsychosocial outcomes amongst NICU infants.

Despite the need, over 50% of mothers with PMADs do not receive adequate treatment. Although evidence-based interventions to prevent and treat PTSD exist, no solution currently exists to provide specialized care for mothers who are exposed to sights, sounds, and situations in the NICU that can provoke trauma responses. A reasonable treatment strategy is teaching parents strategies how to adapt to stressful stimuli in the NICU and to cope with stressors during the early phases of ASD. The mainstay of CBT for PTSD utilizes relaxation techniques, guided imagery, and imaginal exposure. An important treatment limitation has always been the inability for the therapists to directly observe how their patients cope with exposure to stressful stimuli and how these stressors contribute to their symptoms. Immersive mediums such as VR have yet to be used for treating NICU parents with PTSD, or at-risk for PTSD.

Cognitive behavioral therapy (CBT) is a short-term, problem-focused approach that has established efficacy in treating a vast array of clinical presentations, including the conditions common in PMADs. Although the specific aspects of CBT protocols vary, most CBT approaches include key aspects of Beck's cognitive therapy. Therapists who deliver CBT develop an individualized case formulation of each patient's clinical presentation, which in turn guides the selection of specific intervention strategies. Contemporary manifestations of CBT incorporate additional evidence-based intervention strategies, such as those that promote acceptance, mindfulness, and distress tolerance. Results from randomized controlled trials of case formulation-based CBT indicate that it is highly efficacious relative to wait list control and placebo-controlled conditions and outperforms or enhances use of psychotropic medication across 1- to 2-year follow-up periods. CBT has established efficacy for perinatal populations, although only recently has a case formulation-based treatment manual been elucidated in the literature. It is proposed herein that a CBT case formulation-based treatment approach is especially efficacious in the treatment of PMADS because the selection of strategic interventions can be tailored to the current clinical presentation, psychiatric history, and cognitive behavioral vulnerabilities and life circumstances that are unique to each patient. An important treatment limitation has always been the inability for the therapists to directly observe how their patients cope with stressors and how these stressors contribute to their symptoms. Immersive mediums such as virtual reality (VR) have yet to be used for treating NICU parents at high risk for PTSD.

### 2. Innovation

Unlike medical conditions such as pneumonia having distinct physical findings discoverable on examination to help physicians diagnose and treat their patients, management of psychological conditions relies heavily on validated questionnaires and skillful interviews by therapists. Models for structured interviews have been proposed-all intended to help therapists gather reliable data used to diagnose psychological conditions and assess the effectiveness of treatment plans. However, therapists currently have no way to directly observe how their patients cope with stressors, how these stressors contribute to their symptomatology, and whether treatment is working.

The example Virtual Reality platform, COntinUum based extended Reality Assisted coGnitive behavior therapy with Experiential practice (COURAGE), is novel as it provides a personalized virtual psychotherapy experience with experiential practice capabilities. Using the system, practitioners expose patients to virtual reality versions of visual and auditory stressors in a controlled manner during therapy sessions. By observing how their patients react in real time to such exposures, CBT therapists better help their patients manage stress, which reduces depression, fatigue, sleep disturbances, and anxiety.

Virtual reality technology allows for a 360-degree interactive environment for patients to participate in immersive education and therapy experiences (e.g., exposure) and if needed place other avatars of remote participants (peer coaches) into the environment. The example provides approaches for a virtual therapy session where patients are able to, in embodiments, interact with therapists through virtual avatars that provide anonymity to alleviate vulnerability issues and have gesture-based feedback for the therapist/moderator. The patients can be at the hospital or their home and at different stages of recovery, all sharing experiences and learning with the coach. The innovation provides a novel approach for dissemination and implementation of education, psychosocial interventions, and collaboration in a one-on-one setting or in a group across the continuum of care. An aim is to drive greater patient engagement and enhanced learning/practice through guided simulation.

As the technology and headsets are implemented, the platform can be extended to include augmented reality combined with virtual reality as needed for administering the interventions. NICU parents who are at increased risk for acute stress disorder, PTSD and clinical depression require special attention because when untreated, these conditions have negative long-term impact on parents, their relationships, and psychosocial and neurodevelopmental outcomes for their babies. NICU stress triggers associated with maladaptive responses can be made into virtual assets to which therapists can exposure their patients in a controlled, graduated manner, in order to help them adjust and develop and strengthen coping skills, thereby mitigating their risk for adverse mental health outcomes such as PTSD.

Innovation 1: COURAGE's AR/VR technology allows patients and coaches/counsellors/therapists to be immersed in a unique therapy session experience inside a safe virtualized 360-degree interactive environment. In this environment, the therapist can expose the patient to a series of visual and auditory scenarios designed to elicit certain psychological responses. Using COURAGE's rapid scenario delivery system, a therapist can test his/her mental model of the patient's psychological needs very quickly through iterative exposure and observations, and thereby formulate treatment strategies and plans more customized to the patient's needs. The disclosure provides approaches for a therapy session where patients are “living” through a stressful scenario in the safe and comfortable environment that is away from the real stressors. In embodiments, the therapist has full control over the patient's exposure and is able to be constantly communicating with the patient either remotely (using COURAGE's remote access portal), or in-person in the office. The therapist, through their device, is seeing and hearing the virtual therapy session as seen by the patient. In embodiments, the COURAGE platform includes tactic input. An aim is to drive greater patient engagement and enhanced learning/practice through guided simulation. Using the COURAGE tool, therapists can better identify and treat specific situations or conditions that can increase the patient's risk for PTSD, depression, fatigue, sleep disturbances, and anxiety.

Innovation 2: COURAGE gives therapists a new and powerful tool to directly observe, in a therapy session, how their patients cope with stressors, how these stressors contribute to their symptomatology, and whether treatment is working. Unlike medical conditions such as pneumonia having distinct physical findings discoverable on examination, management of psychological conditions relies heavily on validated questionnaires, skillful interviews, and imaginary techniques by the therapists. Models for structured interviews have been proposed, all intended to help therapists gather data used to diagnose psychological conditions and assess the effectiveness of treatment plans. However, these tools only provide therapists with indirect evidence whereas COURAGE allows for direct observation and therapy of the stress triggers in a virtual simulation environment.

Innovation 4: The remote portal capabilities of the system allow therapists and patients to engage in therapy potentially from anywhere in the world. The scenarios presented by the media can be provided in different languages and/or can be considerate of cultural norms. The system has a multi-patient mode that allows for group therapy. COURAGE also helps mental health experts provide services to patients in underserved areas where mental health services are lacking; thereby mitigating risk for preterm births, peripartum morbidity and mortality in these areas.

Innovation 5: The COURAGE system bridges equity gaps. The system works with and across various electronic devices. To engage in a COURAGE therapy session, a patient does not need expensive headsets, but only their cell phone, mobile or WIFI service and a cardboard viewer to convert the phone into a smartphone VR/AR device. As the provision of broadband and cell services in underserved areas becomes more promising, COURAGE is ready to serve these populations.

Innovation 6: COURAGE provides a novel approach to dissemination and implementation of education and collaboration in a one-on-one setting or in a group across the continuum of care. As a virtual simulation tool, the COURAGE platform can be used for educational purposes of medical trainees and staff. The instructor can show various scenarios and provide real time feedback and coaching to the students.

Innovation 7: Video selection heuristic algorithm. As COURAGE is used, information on practitioners' video choices for different psychometric profiles (e.g., with patient information anonymized or de-identified) is collected, which is used to inform a video selection heuristic algorithm with predictable outcomes. In example embodiments, the algorithm is implemented with an AI/ML model. In embodiments, the AI/ML model is a machine learning model that implements a recommendation algorithm, such as a linear regression algorithm used to find the best linear approximation to a data set. In this example, the linear regression algorithm is used to predict how a patient will respond to particular media used in the virtual therapy session. For example, if it is observed that a first patient responds to stress inoculation therapy by being more resilient and exhibiting a lower trauma response compared to without or before the stress inoculation therapy, then the system can identify a predicted effectiveness of the stress inoculation therapy for a second patient if the first patient and the second patient have at least some overlapping features or characteristics in common.

Technology Architecture. In example embodiments, components of the system are developed and/or implemented using the Microsoft® Power Platform® infrastructure. The plug and play, low-code architecture is apt for developing an agile platform in a short timeframe to test and evolve to future versions as needed. It is also HIPAA compliant. The Power Platform® portal provides web-based access for the patient and practitioner to use the system. A low-cost VR headset can be provided to the patient to utilize with their mobile device (see, e.g., FIG. 3B). In embodiments, only the patient wears the VR headset during therapy. A media or video content library, data from ratings, and patient well-being self-reporting is stored in the Dataverse® database. Azure® media services are used as part of the online video viewer platform, in embodiments. The Microsoft Teams® video chat, Zoom® video chat, or other healthcare compliant video chat functionality is used for remote evaluation of the patient by the practitioner and for the practitioner to better administer the validated mental health measures, e.g., using one or more webcams of one or more devices.

The present disclosure provides an all-inclusive virtual CBT psychotherapy solution that enables therapists to teach patients to learn, test, and improve coping skills through exposure and with engagement in treatment across the continuum of care.

### 3. Approach

Practice relevance. Stress due to exposure to the NICU environment experienced by parents of NICU infants is associated with elevated rates of anxiety, depression, and sleep disturbance. A recent study reported that 52% of NICU mothers have elevated stress levels, with estimates of Postpartum Mood and Anxiety Disorders (PMADs) ranging from 28-70%, for mothers of infants hospitalized in a NICU. Prevalence of Post-Traumatic Stress Disorder (PTSD) can be as high as 88% of mothers of NICU infants with congenital defects. PTSD is highly comorbid with depression as well as impaired maternal-infant bonding, attachment, and interaction, which all have been associated with impairments in infant growth and neurodevelopment, which, in turn, are associated with long term adverse biopsychosocial outcomes among NICU infants. Furthermore, adverse parental responses continue beyond the NICU hospitalization. To identify parental stressors in the NICU, a point prevalence assessment of 114 parents was conducted in May-June of 2018. Using the Parental Stress Scale NICU version (PSS: NICU), a well-established self-report instrument asking parents to rate their level of stress associated with 37 items in four categories (i.e., sights and sounds of the NICU, baby's appearance, parental role, staff communication), it was found that 23% of parent responses were either very or extremely stressful across all items.

For this example, a virtual NICU-specific content library of stressors was developed and targeted through Cognitive Behavior Therapy with virtual exposure (vCBT). The aims of the example study are to 1) create and assess the stressfulness and relevance of new and improved VR video scenario choices in this NICU-specific library, and 2) conduct a randomized control pilot to determine to examine the extent to which vCBT can mitigate PTSD symptomatology relative to traditional CBT. Implementation of this proposal involves: a) Development of the NICU-specific VR content library, b) Customization of Cognitive Behavior Therapy for NICU Parents, and 3) Feasibility evaluation and a prospective randomized control pilot study.

### Feasibility Study R&D Plan:

A study team uses the COURAGE system, an AR Enhanced Video Content Library for PSS: NICU, CBT modules customized towards parental stress in the NICU, and a pilot clinical feasibility study.

### A) Create and Test 360-Degree AR/VR Video Library

### Technical Build:

i. Content Library: Of the 37 stressor items in the PSS: NICU, 23 are amendable for video re-enactment. Specifically, 19 are related to the sights and sounds of the unit and the appearance and behavior of infants and 4 are related to staff communication and behaviors. The remaining 14 PSS: NICU items are related to relationship with infant and parental role and other staff behaviors that are best addressed in the conversational aspects of the CBT sessions. An expert team of clinicians, behavior health psychologists, visual and auditory content design and engineers create a series of vignettes capturing each of the 23 stressor items in the PSS: NICU. These vignettes are enacted by neonatal providers using Laerdal baby simulators at a Neonatal Education and Simulation-based Training (NEST) Suite. The trained simulation teams facilitate the scripting and execution of these simulation vignettes. The vignettes are captured and recorded using multipoint 360-degree video technology. The VR/AR team edits and stitches videos to make 23 2-4 minute long 360-degree videos and an AR/VR Graphics consultant superimposes augmented reality objects onto these videos that were impossible to do with real scenes given limitations on the simulator. The psychologist is able to toggle these AR objects during the vCBT session (e.g., alarms flashing and sounding, extra IVs, chest tubes, various ventilators attached to the baby). Each video depicts a specific stressor in the PSS: NICU tool. For example, for item 2 (i.e., Constant noise of monitors and alarms), the scenario shows alarming monitors and ventilators with varying frequency and volume and for item 6 (i.e., Having a machine breathe for my baby), the baby simulator is attached to the ventilator in an Isolette.

### Augmented Virtual Reality Assets

Ventilators (Jet, Oscillator, and Percussive)—next to the warmer, animates graphical loop displays, lights and beeps, alarms
IV Pole with feeding and IV pumps-pumps have displays and displays changing numbers and alarms
Objects and equipment connected to the baby (Chest tube drainage system, peritoneal drainage system, Dialysis machine, ECMO circuit, Auto-Transfusion equipment, Lymphatic drainage system, Ventriculostomy)—have displays and display changing numbers, lights and beeps, and alarms

ii. Emotion Regulation VR content library: To enable the therapist to coach patients on mindfulness and relaxation techniques and help them manage distressing emotions effectively, a content library of immersive 360-degree VR videos with pleasant, relaxing imagery and sounds is created (e.g., beach, mountain).

iii. Library vignette Viewer and control panel: The viewer is a web-based component of COURAGE that allows the video vignettes to be played and controlled by the psychologist. Psychologists, using a tablet or laptop, use the viewer web-application to operate COURAGE remotely with a virtual therapy management session; example uses include logistical functionalities, select and show video vignettes and its augmented assets. Patients use a mobile app viewer to sign on to COURAGE, interact with the psychologist through a secure telemedicine channel, and navigate through the vignettes guided by the psychologist. They can use their own smartphone devices placed inside cardboard viewers to navigate the viewer, for example, as shown at FIG. 3B.

B) Customize Cognitive Behavior Therapy for Stress Reduction: A team of clinical psychologists and Neonatologists manualize content for the CBT treatment that includes 6 sessions for the purpose of NICU stress reduction, with special focus on items reported to be most associated with post-traumatic stress symptoms. Session 1 includes introduction to relaxation training for anxiety reduction (e.g., deep breathing, progressive muscle relaxation) and subsequent sessions involve imaginal exposure to NICU-related stressors paired with relaxation. The program incorporates additional evidence-based intervention strategies, such as those that promote acceptance, mindfulness, and distress tolerance. The vCBT sessions progress similarly to the CBT session with the following distinctions. The team develops an initial individualized case formulation using each patient's baseline IES results (assessed prior to the start of treatment), which in turn guides the selection of module content and VR asset exposures during the vCBT sessions. The selected VR scenarios (e.g., baby A connected to a ventilator) are used to deliver the exposure for the vCBT sessions to reduce anxiety/distress, regulate emotions, and promote acceptance. In addition, by learning how the participant reacts to the exposure, the therapist makes therapeutic adjustments in real time. The IES results in week two give the therapist added information to further customize the case formulations.

### C) Assessment of COURAGE AR Video Library

Subject Recruitment. The study team explains the study and offers patients who meet inclusion criteria an opportunity to view and evaluate videos in the library. Mothers interested in participating are consented in concordance with an institutional review board. For example, 30 participants can be enrolled. Inclusion criteria. Mothers of infants admitted to the NICU; aged 18-50 years old. Exclusion criteria. Parents with other pre-existing mental health conditions determined to require extensive psychosocial intervention by a psychosocial team. These parents are treated separately outside of the study.

Methods: Study coordinator screens mothers of babies in the NICU within 1 week of admission with the IESR tool. Mothers meeting inclusion criteria are asked to complete an IES-R assessment tool and then view and evaluate the stressfulness and relevance of 10 videos in the library that are both matched and unmatched to their personal NICU experiences using the Subjective Units of Distress Scale (SUDS) in order to rate VR scenario impact at inducing distress. The study coordinator uses a randomization tool to dictate the sequence of videos presented to subjects and follows the script in presenting each video to the subjects. Each participant evaluating the video content is also evaluated by a psychotherapist overseeing the video session.

D) Clinical Feasibility and Impact Pilot Study: A prospective randomized control study to determine feasibility of the COURAGE platform and its impact on mitigating PTSD precursors and the risk for developing PTSD when used with Cognitive Behavior Therapy for parental stress, depression, anxiety, fatigue, and sleep patterns.

Month 1. Complete hire and orientation of study coordinator, organize and develop detailed project plan with study and technology development team.

Month 6-11. Recruitment and Prospective randomized control study

Month 11-12. Results analytics and report preparation.

Subject Recruitment. The study team offers parents (fathers and mothers) who meet inclusion criteria an opportunity to participate in the COURAGE Study. Parents interested in participating are consented in concordance with a hospital institutional review board. As described below in the sample size estimate section, it is anticipated that enrolling 20 subjects in the control arm (traditional CBT), and 20 subjects in the intervention arm (vCBT) is envisioned. Inclusion criteria. Mothers (of babies admitted to the NICU) who screen positive for PTSD symptoms (IES-R score ≥24). Parents of babies anticipated to remain in the NICU for at least 4 weeks. Exclusion criteria. Parents screening positive for PTSD symptoms and have other pre-existing mental health conditions determined to require extensive psychosocial intervention by a psychosocial team. These parents are treated separately outside of the study.

Sample size estimates. An example NICU admitted 1705 babies in 2020. In a sample of 156 NICU mothers screened with the Impact of Events Scale-Revised (IES-R), 34% scored positive for PTSD symptoms (≥24). In 2020, 316 babies stayed at least 4 weeks or more, which estimates 52 mothers (34% of 316) meet inclusion criteria in the study period (6 months). Assuming an 80% study enrollment rate, it is estimated that 40 parents would agree to participate in this study over 6 months (20 in control group receiving CBT and 20 in intervention group receiving vCBT). In a recent pilot study of 9 patients, the average IES-R score in parents who screened positive was 53+/−18. Therefore, the study has adequate sample sizes (20 required for each group) to detect an 11-point reduction in the intervention group receiving virtual enhanced CBT (vCBT) with a confidence of 95% and power of 0.8. Although the sample size calculation allows for a 20% attrition rate, very low attrition is expected since babies may be hospitalized in the NICU for the entire study period.

Institutional Review Board (IRB). Study design and protocols are written and submitted for approval by a hospital institutional review board in accordance with human subject protection requirements set forth by the institution and NIH.

### Method:

Randomization. All subjects enrolled into the study receive 2 CBT sessions per week with a NICU psychologist, up to a maximum of 4 weeks, depending on when a baby is discharged from the NICU. At the beginning, each subject is randomized to the control group (CBT) or intervention group (vCBT) arm, which dictates the type of CBT they receive for the duration of the NICU hospitalization. The same psychologist can administer all CBT sessions.

Cognitive Behavior Therapy Session. Before CBT sessions begin, subjects complete a baseline set of psychometric assessment tools (Impact of Events Scale-Revised (IES-R); Center for Epidemiologic Studies-Depression (CES-D) tool; PROMIS Questionnaires for anxiety, sleep, and fatigue and Parental Stress Scale) on enrollment. In weeks 2-4, each subject may receive 2 therapy sessions per week with a therapist (with or without virtual assets depending on the arm they randomize to). The patient's heart rate is monitored and recorded throughout all CBT sessions. The intervention group uses VR relaxation training, guided imagery, and exposure to AR enhanced video vignettes administered by psychotherapists using the COURAGE online VR viewer. The control group receives a scripted CBT program matching vCBT without the video exposure. The scripted CBT program and selection of video vignettes from the library are customized to address items the subject identified as very or most stressful in their PSS assessments. Subjects are asked to complete an IES-R at the beginning and end of weeks 2 and 3. Before discharge or at the end of week 4, the study coordinator administers a final set of psychometrics tools, the Telemedicine Usability Questionnaire and exit interviews for qualitative feedback from each subject. This study design yields 40 IES-R time series datasets obtained over 4 weeks of therapy (20 in each arm). In addition, datasets may be obtained and analyzed from subjects completing fewer therapy sessions if their baby is discharged earlier. It is estimated that minimal attrition rates occur since babies may be admitted to the NICU for the entire duration of the study.

D.1) Feasibility Monitoring and evaluation. Logistical feasibility of administering CBT sessions in the NICU is assessed by session date and time stamps, the frequency and reasons for missed and rescheduled therapy sessions, include those related to NICU and family. Study teams track frequency and type of technical difficulties and resolutions. Subjects and Psychologist provide feedback on COURAGE's usability via an adapted version of the Telehealth Usability Questionnaire. The NET Promoter Score assesses the psychologist's and subject's likelihood to recommendation vCBT and CBT sessions. All results are summarized with descriptive statistics in an executive report.

TABLE
Study Data Record Strategy

| | | vCBT (AR enhanced video |
|---|---|---|
| | CBT (guided imagery) | vignettes) |
| Week 1 | Subject: IES-R, CES-D, PSS: NICU, | Subject: IES-R, CES-D, PSS: NICU, |
| (within 5 | PROMIS | PROMIS |
| days of | Staff: logistical feasibility, technical | Staff: logistical feasibility, technical |
| Admission) | issues log, Patient acuity score | issues log, Patient acuity score |
| Week 2 | Subject: IES-R X 2 (before CBT # 1 | Subject: IES-R X 2 (before vCBT # |
| (CBT | and after CBT # 2) | 1 and after vCBT # 2) |
| session # 1 | Staff: logistical feasibility, technical | Staff: logistical feasibility, technical |
| and 2) | issues log, Patient acuity score | issues log, Patient acuity score |
| Week 3 | Subject: IES-R X 2 (before CBT # 3 | Subject: IES-R X 2 (before vCBT # |
| (CBT | and after CBT # 4) | 3 and after vCBT # 4) |
| session # 3 | Staff: logistical feasibility, technical | Staff: logistical feasibility, technical |
| and 4) | issues log, Patient acuity score | issues log, Patient acuity score |
| Week 4 | Subject: IES-R, CES-D, PSS: NICU, | Subject: IES-R, CES-D, PSS: NICU, |
| (CBT | PROMIS | PROMIS |
| session # 5 | Staff: logistical feasibility, technical | Staff: logistical feasibility, technical |
| and 6) | issues log, Patient acuity score, Net | issues log, Patient acuity score, Net |
| | Promoter score, Exit interview | Promoter score, Exit interview |

D.2) Impact of vCBT on LES-R and other psychometric measures. In each arm, each week yields 20 sets of psychometric scores (in particular IES-R), totaling 80 for the entire arm and 160 for both arms. Six sets of Interval changes in IES-R scores between weeks amongst subjects in the vCBT arm are compared to those in the CBT arm. CES-D, PSS: NICU and PROMIS scores in the control and intervention groups are summarized and compared. Distribution patterns determine the most appropriate statistical approach (parametric vs. non-parametric). T-test or Wilcoxon-Rank Sum test analyzes average changes between control and intervention groups. The paired T-test or Wilcoxon-Rank Sum test analyzes changes over time intervals within each group. A patient acuity score is calculated and recorded for each NICU patient in the electronic medical record daily. For each subject, their baby's average acuity score on the day of the CBT session is recorded and used to risk adjust the psychometric scores.

An objective of the Phase I feasibility clinical randomized control study is to estimate vCBT's impact on trauma/PTSD, stress, depression, sleep, anxiety, and fatigue, which estimates a-priori values for a power calculation in the phase 2, clinical trial. The commercialization potential is immense, the addressable market for PMADs including PTSD is 16 billion; globally 20% of mothers are impacted, per WHO. This innovative tool, delivering therapeutic experiential practices, paves a novel and disruptive path not only to treat trauma experienced by parents of infants in the NICU, but also other psychological conditions.

### Recruitment and Retention Plan

Subjects are recruited from parents whose children have been admitted to NICU, in the Newborn/Infant Intensive Care Unit (N/IICU). The subject mothers are approached on the 5th day in the NICU. RAs and staff help in identifying those who meet the basic eligibility criteria. Those subject mothers that meet eligibility may be provided information about the study, a consent form and initial IES-R assessment to gauge eligibility for the study. In an effort to increase retention, one can work to schedule the assessments and intervention in the N/IICU and in conjunction with the patients' visiting schedule. It is hoped to minimize the burden of participation with this procedure. To decrease scheduling challenges due to siblings who need childcare, another organization can assist in organizing a volunteer to interact with the sibling while their parent is either completing assessment forms or participating in a CBT or vCBT session. Reminder texts or emails are sent for parents as an alert to upcoming meetings.

Planned Recruitment Activities. Mothers who have a child or children in the N/IICU are approached by the Research Coordinator/Assistant (RA), and/or other staff and offered the opportunity to participate in this program. Staff assist the research team in identifying families who meet the eligibility criteria. Once identified, the RA finds a private area to fully explain the study through the consenting process with the potential participant. The RA reviews the consent process, asks the participant about the procedures in an effort to make sure she understands, answers any questions, and if agreed, has the participant sign the consent/assent. This process should take approximately 10-15 minutes. All participants who consent to participation receive a copy of the signed consent form. A copy is placed in their chart. Participants are informed that their decision to participate in this research may not affect the care that their child (ren) receives at the NICU. A combined consent-authorization document is used. Parents provide consent for their child to assess information from his/her medical record, as children in this study may be infants and therefore not capable of providing assent. To be able to support the parents in a targeted way, it is important to know date of birth. Since this information is in the infant's chart in the NICU, parental consent is obtained.

### Study Timeline

The timeline for the Phase 1 Clinical Feasibility Pilot Study with Aims 1 and 2 is as follows:

Month 3-4, Aim 1: AR/VR 360-degree. Videos Evaluation. A feasibility study is conducted to assess the stressfulness and relevance of new and improved 360-degree video scenario choices in the COURAGE AR/VR library. The new content is developed based on criteria from the Parental Stress Scale, NICU version (PSS: NICU). Parents of NICU infants (N=30) are asked to view and evaluate 20 new videos content that is both matched and unmatched to their personal NICU experiences using (the scores from the PSS: NICU). Parents then use Subjective Units of Distress Scale (SUDS) to rate AR/VR scenario impact at inducing distress. Each parent evaluating the video content may be evaluated by a psychotherapist overseeing the video session.

Month 5-10, Aims 2: Randomized Control Trial. The randomized controlled trial involves 40 NICU parents having moderate to high PTSD symptoms (IES-R >=24). Parents are randomly assigned to either a vCBT group or CBT group and each receives 6 sessions over 4 weeks with first week for recruitment and weeks 2-4 with 2 psychotherapy sessions per week. The vCBT uses relaxation training, guided imagery, and exposure to NICU-specific VR scenarios administered by psychotherapists using the COURAGE online VR viewer. The CBT group receives a scripted CBT program matching vCBT without the video exposure. PTSD, depression, anxiety, maternal attachment, fatigue, and sleep disturbances are evaluated, as well as attrition rate, usability, reliability, patient engagement, satisfaction, and overall perceived value.

Enrollment Rate per month. The estimated enrollment rate per month is at least 15 for third and fourth months for Aim 1. For Aim 2, it is at least 7 each month for the randomized controlled trial with 40 subjects over 6 months with each subject getting CBT or vCBT for 4 weeks. Based on prior research data at the NICU, patients with IES-R scores >=24 are 33% of the total patient population. The RI inpatient population last year with 4 weeks length of stay or more for 6 months was 158 and 33% of that is 52 patients. Assuming 80% enrollment, approximately 41 patients over 6 months, which is more than the goal of 40 patients. The overall example project timeline is shown below (X indicates target month for completion).

| | YEAR 1 (months) | | | | | | | | | | | |
|---|---|---|---|---|---|---|---|---|---|---|---|---|
| PROJECT TIMELINE | 1 | 2 | 3 | 4 | 5 | 6 | 7 | 8 | 9 | 10 | 11 | 12 |
| Aims 1: Develop CBT script and 20 | X | X | | | | | | | | | | |
| AR/VR scenario 360 deg. videos with | | | | | | | | | | | | |
| online viewer portal | | | | | | | | | | | | |
| Aims 1: 30 Subjects score 20 videos | | | X | X | | | | | | | | |
| for 600 datapoints | | | | | | | | | | | | |
| Aims 2: Randomized Control Trial | | | | | X | X | X | X | X | X | | |
| with 40 subjects, 20 with CBT vs. 20 | | | | | | | | | | | | |
| with vCBT | | | | | | | | | | | | |
| Aims 2: Data Analysis and Reporting | | | | | | | | | | | X | X |

### Protection of Human Subjects

This Human Subjects Research meets the definition of Clinical Research.

### Risks to Human Subjects

### Human Subjects Involvement and Characteristics

This example is provided as a prospective control study. Study participants are 40 adult subjects (ages 18-50) who are mothers of babies admitted to a quaternary Neonatal Intensive Care Unit. Women may be represented in this study population. African American and Latino minority subjects may also be accrued whenever possible. Children, however, may not be enrolled in this study as by definition they do not meet the inclusion criteria. All human subject research-related activity may occur at the hospital Newborn/Infant Intensive Care Unit (NI/ICU) within a Division of Neonatology. Subject recruitment may take place within the Division of Neonatology. All Cognitive Behavior Therapy sessions may be administered to subjects at a hospital by psychologists qualified to perform virtual Cognitive Behavior Therapy (vCBT) and Cognitive Behavior Therapy (CBT). All collected data is to be utilized for research purposes. Once the data have been gathered, patient/subject identities are encrypted so that such information may be available only to the PI and study team members. The relevant data may be analyzed by PIs within the Division of Neonatology. The study team may offer mothers who meet inclusion criteria an opportunity to participate in the study. Mothers interested in participating may be consented in concordance with a hospital institutional review board. As described below in the sample size estimate section, we anticipate enrolling 20 subjects in the control arm, and 20 subjects in the intervention arm.

Inclusion Criteria: (i) Age 18-50 years. (ii) Mothers with baby(s) admitted to the NI/ICU with baseline IES-R score equal or greater than 24 and verified by the psychosocial team to have mild to moderate PTSD symptoms, (iii) Mothers of babies anticipated to remain in the NI/ICU for at least 4 weeks.

Exclusion Criteria: (i) Age <18 or >50 years. (ii) Mothers with baby(s) admitted to the NI/ICU with baseline IES-R score less than 24 or with other pre-existing mental health conditions determined to require extensive psychosocial intervention by a psychosocial team, (iii) Mothers of babies anticipated to remain in the NICU for less than 4 weeks. Sources of Materials Study staff may collect data from the electronic medical record for research purposes to include subject's baby's name and medical record number (MRN); subject's name, age, gender, and ethnicity. Study files may be accessed to retrieve subject's IES-R, CES-D, PROMIS and PSS: NICU assessment results, dates of study, Virtual Cognitive Behavior Therapy session dates and subject responses to virtual assets.

### Potential Risks are Minimized

The subject's exposure to virtual stress factors during the vCBT and CBT sessions could have psychological risks for negative affective states such as anxiety, depression, sleep disturbances, fatigue and altered behavior. Since such exposures are conducted in the presence of a trained psychologist who can intervene immediately, the risk for harm is greatly minimized and is outweighed by the potential benefits subjects can experience via the vCBT or CBT sessions that they would otherwise not receive as shown in an initial feasibility pilot over a smaller number of subjects. The COURAGE tool used by the subjects in the intervention group is HIPAA compliant. In this way, the low to moderate risk posed to the enrolled subjects is minimized. In the event of adverse effects to subjects, it is important to ensure necessary medical or professional intervention through a data safety monitoring plan.

### Potential Benefits of the Research to Human Subjects and Others

For subjects, the anticipated benefits from receiving vCBT or CBT over 4 weeks at 2 times per week for total 6 sessions from weeks 2-4, outweighs the psychological risk of exposure to virtual stress stimuli during psychotherapy which is simply mimicking the day-to-day stressors parents are already going through in the NICU and not injecting additional stressors. The initial feasibility pilot has shown that most parents benefitted from this approach and found it to be useful and engaging. Furthermore, their participation helps the future care of parents whose babies are admitted to a NICU. In addition, the subjects in this study are exposed to low to moderate risk to include possible breach of privacy and loss of confidentiality. This risk is minimized through use of study ID numbers and de-identified data sheets as described in the “Protection against Risk” section above. Therefore, the risk posed to the enrolled subjects is reasonable in relation to the importance of the knowledge to be gained from this study.

### Importance of the Knowledge to be Gained

By working together in this multi-disciplinary setting, the investigators improve the management and outcomes of parental stress in a NICU. As this example poses no greater than low to moderate breach of confidentiality risk to the subject population and this is minimized through use of study ID numbers and de-identified data sheets, the risk posed to the enrolled subjects is reasonable in relation to the importance of the knowledge to be gained from this study. As this study poses some psychological risk that is largely mitigated by having the psychologist ready to intervene during the sessions, the psychological risk posed to the enrolled subjects is reasonable in relation to the importance of the knowledge to be gained from this study.

### 4.1. Study Design

4.1.a. Detailed Description

Perinatal Mood & Anxiety Disorders (PMADs) are the most common complication of childbirth with an economic impact upwards of $14 billion. Close to 15% of postpartum women in the U.S. suffer from PMADs annually, with 28-70% for mothers of infants hospitalized in a Neonatal Intensive Care Unit (NICU). In addition, according to the World Health Organization (WHO), 20% of mothers worldwide are similarly affected with PMADs. This population is at particular risk for Post-Traumatic Stress Disorder (PTSD) with prevalence as high as 88% of mothers (66% of fathers) of infants with congenital defects, relative to 5.6% for the general population of mothers following childbirth. Within the first 3-5 days of admission, 32% of parents experience onset of Acute Stress Disorder (ASD) which places them at risk for PTSD. In cases of infant death, 35% of mothers continue to have clinical PTSD thirteen months later. Moreover, PTSD is highly comorbid with depression as well as impaired maternal-infant bonding, attachment and interaction which all have been associated with impairments in infant growth and neurodevelopment. These problems in turn are associated with long term adverse biopsychosocial outcomes amongst NICU infants. Despite the need, over 50% of mothers with PMADs do not receive adequate treatment. Although evidence-based interventions to prevent and treat PTSD exist, no market solution currently exists to provide specialized care for mothers who are exposed to sights, sounds, and situations in the NICU that can provoke trauma responses.

Aim 2 of this example is a randomized controlled trial with 40 NICU parents having moderate to high PTSD symptoms (IES-R >=24). Parents are randomly assigned to either a vCBT group or CBT group and each receive 6 sessions, 2 per week over 4 weeks. The vCBT uses relaxation training, guided imagery, and exposure to NICU-specific AR/VR scenarios administered by psychotherapists using the COURAGE online AR/VR viewer. The CBT group receives a scripted CBT program matching vCBT without video exposure. PTSD, depression, anxiety, maternal attachment, fatigue, and sleep disturbances are evaluated, as well as attrition rate, usability, reliability, patient engagement, satisfaction, and overall perceived value. These aims are executed by investigators from the hospital. Results help estimate a priori values of expected psychometric change for a power calculation for a phase 2 future clinical trial. Administration of COURAGE vCBT reduces PTSD symptoms and thereby mitigates development of PTSD, a step in demonstrating efficacy in this high-risk population.

The 12-month project begins with CBT script development and VR content generation in months 1-2. It continues with parents who have an infant in the NICU viewing and offering feedback to the newly created VR scenarios in months 3-4. In months 5-10, 40 parents are equally randomized into a vCBT intervention group and CBT control group. The primary focus is on trauma, a risk factor of PTSD. In addition, anxiety, fatigue, depression, and sleep disturbances are measured using validated tools. Mothers whose babies are anticipated to remain in the NICU for at least 4 weeks, and who are evaluated to have moderate to high PTSD symptoms are enrolled. For each subject, the Research Assistant may administer the IES-R to determine eligibility for the study. The PSS: NICU, PROMIS, CES-D Questionnaires may also be administered for anxiety, sleep, and fatigue. The IES-R measure is administered 1 to 2 times per week over the course of the program; week 1 before session 1; week 2, before session 2 and after session 3; week 3, before session 4 and after session 5; week 6, after session 6. This builds a base profile of the subjects that may be monitored over 4 weeks. A highly qualified CBT psychotherapist provides each subject with eight, 60 minute, vCBT or CBT psychotherapy sessions. In months 11-12, data analysis and reporting occur.

4.1.b. Primary Purpose Prevention
4.1.c. Interventions

| Type | Name | Description |
|---|---|---|
| Behavioral | Virtual Reality | A team consisting of Psychologists, |
| (e.g., | Assisted Cognitive | Neonatologists, and psychotherapists will provide |
| Psychotherapy, | Behavior Therapy | vCBT using relaxation training, guided imagery |
| Lifestyle | | and exposure to NICU-specific AR/VR scenarios |
| Counseling) | | using the Vital Start COURAGE AR/VR portal. |
| | | Psychotherapists will have the ability to expose |
| | | their subjects to these items in the course of 6 |
| | | sessions of vCBT therapy (the intervention). |
| | | These CBT sessions will be customized by the |
| | | psychotherapists according to each subject's |
| | | initial PSS: NICU results taken the previous |
| | | session. |

### 4.2 Outcome Measures

| Type | Name | Time Frame | Brief Description |
|---|---|---|---|
| Primary | IES-R Score | Week 1 before | Impact of Events Scale Revised |
| | | session 1; | (IES-R) measures the effect of |
| | | week 2, before | routine life stress. It is a |
| | | session 2 and | validated, 22 item, self-report, |
| | | after session 3; week | 5-point Likert type scale. |
| | | 3, before | Responses range from 0 (not at |
| | | session 4 and after | all) to 4 (extremely). While not |
| | | session 5; | a diagnostic tool, it is able to |
| | | week 6, after session | measure the subjective |
| | | 6 | responses to traumatic events |
| | | | specifically-intrusion, |
| | | | avoidance, and hyperarousal. |
| | | | The IES-R can generate a score |
| | | | between 0 and |
| | | | 88. A total score of 24 or |
| | | | higher is concerning for post- |
| | | | traumatic stress disorder. |
| Secondary | Ps, proportion of | Week 1 (baseline | The PSS: NICU assessment tool |
| | very stressful or | assessment) and | is a validated self reporting tool |
| | extremely stressful | week 4 (final | that ask NICU parents to rate 37 |
| | responses in the | assessment) of the | items on a 5-point Likert type |
| | Parental Stress | subject's enrollment | scale (Not stressful at all, A |
| | Scale Assessment | period. | little stressful, Moderately |
| | (PSS: NICU) | | stressful, Very stressful, |
| | | | Extremely stressful). |
| Secondary | Subjective Units of | Week one, before | The Subjective Units of |
| | Distress Scale | session 1 of subjects | Distress Scale (SUDS) is a |
| | (SUDS) | enrollment period | self-assessment tool rated on |
| | | | a scale from 0 to 100, 0 |
| | | | feeling perfectly relaxed and |
| | | | 100 feeling the worst anxiety |
| | | | or distress one can imagine. |
| | | | It is used to measure the |
| | | | intensity of a persons |
| | | | distress, anxiety, or |
| | | | nervousness. Once |
| | | | established, this can be used |
| | | | to gauge different areas of |
| | | | distress or disturbance that |
| | | | require attention. In this scale, |
| | | | the psychotherapist will establish |
| | | | this scale with each parents |
| | | | before beginning the work. |
| Secondary | CES-D Score | Week 1 (baseline | The Center for Epidemiologic |
| | | assessment) and | Studies Depression Scale (CES- |
| | | week 4 (final | D) is designed to measure |
| | | assessment) of the | depressive symptomatology. It |
| | | subject's enrollment | was developed for use in |
| | | period. | epidemiologic studies of |
| | | | depression in the general |
| | | | population. The 20 item scale |
| | | | contains items that address the |
| | | | major components of depressive |
| | | | symptomatology. The CES-D |
| | | | can generate a score between 0 |
| | | | and 60. |
| Secondary | PRO(sd-8) score, | Week 1 (baseline | PROMIS was developed by a |
| | the PROMIS short- | assessment) and | consortium of researchers |
| | form instrument for | week 4 (final | working at 12 primary research |
| | Sleep Disturbance | assessment) of the | sites to provide a reliable and |
| | (8 items) | subject's enrollment | valid set of self-reported health |
| | | period. | measures that can be used by |
| | | | researchers to assess health in 3 |
| | | | primary areas and 7 subdomains |
| | | | (Physical health: symptoms, |
| | | | function; Mental Health: affect, |
| | | | behavior, cognition; Social |
| | | | Health: relationship, function). |
| | | | The PROMIS instruments have |
| | | | been tested extensively to be |
| | | | reliable and valid across diverse |
| | | | populations. The PROMIS |
| | | | Instrument used for this study |
| | | | on Sleep Disturbances (8 items) |
| | | | have good correlation with the |
| | | | well-standardized Pittsburgh |
| | | | Sleep quality Index. |
| Secondary | PRO(f-7) score, | Week 1 (baseline | PROMIS was developed by a |
| | the PROMIS | assessment) and | consortium of researchers |
| | short-form | week 4 (final | working at 12 primary research |
| | instrument for | assessment) of the | sites to provide a reliable and |
| | Fatigue (7 items) | subject's enrollment | valid set of self-reported health |
| | | period. | measures that can be used by |
| | | | researchers to assess health in 3 |
| | | | primary areas and 7 subdomains |
| | | | (Physical health: symptoms, |
| | | | function; Mental Health: affect, |
| | | | behavior, cognition; Social |
| | | | Health: relationship, function). |
| | | | The PROMIS instruments have |
| | | | been tested extensively to be |
| | | | reliable and valid across diverse |
| | | | populations. |
| | | | The PROMIS Instruments |
| | | | used for this study on Fatigue |
| | | | (7 items) have good |
| | | | correlation with the well- |
| | | | standardized FACIT-Fatigue |
| | | | Scale. |
| Primary | PRO(a-7) score, | Week 1 (baseline | PROMIS was developed by a |
| | the PROMIS | assessment) and | consortium of researchers |
| | short-form | week 4 (final | working at 12 primary research |
| | instrument for | assessment) of the | sites to provide a reliable and |
| | Anxiety (7 items) | subject's enrollment | valid set of self-reported health |
| | | period. | measures that can be used by |
| | | | researchers to assess health in 3 |
| | | | primary areas and 7 subdomains |
| | | | (Physical health: symptoms, |
| | | | function; Mental Health: affect, |
| | | | behavior, cognition; Social |
| | | | Health: relationship, function). |
| | | | The PROMIS instruments have |
| | | | been tested extensively to be |
| | | | reliable and valid across diverse |
| | | | populations. |
| | | | The PROMIS Instruments |
| | | | used for this study on Anxiety |
| | | | (7 items) have good |
| | | | correlation with the well- |
| | | | standardized Mood and |
| | | | Anxiety Symptom |
| | | | Questionnaire. |
| Secondary | Delta(Ps, CES-D, | Week 1 (baseline | Changes in Ps, CES-D, IES-R, |
| | IES-R, | assessment) and | and PROMIS scores over 4 |
| | PROMIS scores) | week 4 (final | weeks (week 4 through week 1) |
| | | assessment) of the | |
| | | subject's enrollment | |
| | | period. | |
| Secondary | Technology | Administered to | 7 questions taken and modified |
| | Usability Score | psychotherapist and | from a validated Telemedicine |
| | | subject after each | Usability Questionnaire: |
| | | vCBT therapy | section on ease of use and |
| | | session | learnability (eg. “It was simple |
| | | | to use this system”). Response |
| | | | options is a |
| | | | 5-point Degree of Agreement |
| | | | Likert Scale. Included in the |
| | | | score is the study coordinator's |
| | | | recording of number and type of |
| | | | technical difficulties. |
| Secondary | Percent of completed | Duration of study | Number of completed vCBT |
| | vCBT Appointments | | appointments/number of |
| | | | scheduled vCBT |
| | | | appointments. |
| Secondary | Net Promoter Score | Administered to | Single question asking how likely |
| | | subject in the | the responder would use the |
| | | intervention group | technology (0-10 scale). |
| | | at the conclusion | |
| | | of the 3 week | |
| | | enrollment period | |
| Secondary | Qualitative opinion | At the conclusion | Scripted interview containing |
| | on vCBT sessions | of the 4 week | open-ended questions to elicit |
| | | enrollment period | subject's opinion on what they |
| | | | liked about the vCBT, what can |
| | | | be improved. |
| Secondary | Attrition Rate | Duration of pilot | Number of subjects who did not |
| | | study | complete study (either all the |
| | | | required assessment surveys |
| | | | or vCBT sessions in control and |
| | | | intervention groups)/Number of |
| | | | subjects enrolled. |

### Statistical Design and Power

Sample size estimates. A study at the NICU to screen for the Impact of Events Scale (IESR) found 33% of mothers scored >=24 (moderate to high PTSD). Previous data estimates show that 316 admitted babies stay in the NICU for at least 4 weeks in a year. Assuming an 80% enrollment rate, it is estimated that at least 40 parents would agree to participate in this study over the study period (20 in arm 1 receiving CBT and 20 in intervention arm receiving VR enhanced CBT).

Significance: Therefore, the example study is a feasibility and phase 1 non-powered, pilot study that builds and uses COURAGE in a 6 sessions/4-week CBT psychotherapy program for parents with moderate to high PTSD in the N/IICU. Study subject mothers receive equal monetary compensation for their participation in both the randomly controlled arms of the study. It is hypothesized that the program mitigates PTSD symptoms for those at risk. The aim of this proposal's study design is to estimate reduction in PTSD as well as estimate changes in depression, stress, sleep, anxiety, and fatigue in a cohort of study parents. Results help estimate a priori values for expected change to be used in a power calculation for phase 2, clinical trial.

### APPENDIX: INSTRUMENTS

PROMIS short-form instruments for Sleep disturbances, Fatigue, and Anxiety. PROMIS was developed by a consortium of researchers working at 12 primary research sites to provide a reliable and valid set of self-reported health measures that can be used by researchers to assess health in 3 primary areas and 7 subdomains (Physical health—symptoms, function; Mental Health—affect, behavior, cognition; Social Health—relationship, function). The PROMIS instruments have been tested extensively to be reliable and valid across diverse populations. The PROMIS Instruments used for this study are focused on Sleep Disturbances (8 items), Fatigue (7 items), and Anxiety (7 items); respectively, each have good correlation with existing well-standardized measures—Pittsburgh Sleep quality Index, FACIT—Fatigue Scale, Mood and Anxiety Symptom Questionnaire (Table 2).

IES-R: Impact of Events Scale-Revised (IES-R), Table 3. The IES-R measures the effect of routine life stress. It is a validated, 22 item, self-report, 5-point Likert type scale. Responses range from 0 (not at all) to 4 (extremely). While not a diagnostic tool, it is able to measure the subjective responses to traumatic events, specifically intrusion, avoidance, and hyperarousal. The IES-R score ranges between 0 and 88. A total score of 24 or higher indicates high risk for PTSD.

CES-D: The Center for Epidemiologic Studies-Depression Scale (CES-D), Table 4. The CES-D is a 20-item scale designed to assess depressive symptomatology. It was developed for use in epidemiologic studies of depression in the general population.

Parental Stress Scale: Parental Stressor Scale-NICU (PSS: NICU), Table 5. The PSS: NICU is designed to measure parental perception of stressors related to the physical and psychosocial environment of the NICU. The assessment measures alterations in parental roles, stress related to the sights/sounds of the NICU, and stress related to infant behavior and appearance, and experience with staff communication. The response options for the 5-point Likert type scale are “Not stressful at all, A little stressful, Moderately stressful, Very stressful, Extremely stressful.”

TABLE 1
Technology function and stability assessment tool

| | | | |
|---|---|---|---|
| Date | Type of failure | Description | Resolution |
| | (video, sound, | details | |
| | connectivity) | | |

TABLE 2
Impact of Event Scale Items (Not at All, A Little
Bit, Moderately, Quite A Bit, Extremely)

| Item No. | |
|---|---|
| 1 | Any reminder brought back feelings about it. |
| 2 | I had trouble staying asleep |
| 3 | Other things kept making me think about it. |
| 4 | I felt irritable and angry. |
| 5 | I avoided letting myself get upset when I thought about it or was reminded of |
| | it. |
| 6 | I thought about it when I didn't mean to. |
| 7 | I felt as if it hadn't happened or wasn't real. |
| 8 | I stayed away from reminders about it. |
| 9 | Pictures about it popped into my mind. |
| 10 | I was jumpy and easily startled. |
| 11 | I tried not to think about it. |
| 12 | I was aware that I still had a lot of feelings about it, but I didn't deal with |
| | them. |
| 13 | My feelings about it were kind of numb. |
| 14 | I found myself acting or feeling as though I was back at that time. |
| 15 | I had trouble falling asleep. |
| 16 | I had waves of strong feelings about it. |
| 17 | I tried to remove it from my memory. |
| 18 | I had trouble concentrating. |
| 19 | Reminders of it caused me to have physical reactions, such as sweating, |
| | trouble breathing, nausea, or a pounding heart. |
| 20 | I had dreams about it. |
| 21 | I felt watchful or on-guard |
| 22 | I tried not to talk about it. |

TABLE 3
PROMIS Items

| | |
|---|---|
| Anxiety | |
| I felt fearful | Never, Rarely, Sometimes, Often, |
| | Always |
| I felt anxious | Never, Rarely, Sometimes, Often, |
| | Always |
| I felt worried | Never, Rarely, Sometimes, Often, |
| | Always |
| I found it hard to focus on anything other than | Never, Rarely, Sometimes, Often, |
| my anxiety | Always |
| I felt nervous | Never, Rarely, Sometimes, Often, |
| | Always |
| I felt uneasy | Never, Rarely, Sometimes, Often, |
| | Always |
| I felt tense | Never, Rarely, Sometimes, Often, |
| | Always |
| Sleep disturbances | |
| In the past 7 days | |
| My sleep was restless | Not at all, a little bit, somewhat, |
| | quate a bit, very much |
| I was satisfied with my sleep | Not at all, a little bit, somewhat, |
| | quate a bit, very much |
| My sleep was refreshing | Not at all, a little bit, somewhat, |
| | quate a bit, very much |
| I had difficulty falling asleep | Not at all, a little bit, somewhat, |
| | quate a bit, very much |
| I had trouble staying asleep | Never, Rarely, Sometimes, Often, |
| | Always |
| I had trouble sleeping | Never, Rarely, Sometimes, Often, |
| | Always |
| I got enough sleep | Never, Rarely, Sometimes, Often, |
| | Always |
| My sleep quality was | Very poor, poor, fair, good, very good |
| Fatigue | |
| In the Past 7 days | |
| How often did you feel tired? | Never, Rarely, Sometimes, Often, |
| | Always |
| How often did you experience extreme | Never, Rarely, Sometimes, Often, |
| exhaustion? | Always |
| How often did you run out of energy? | Never, Rarely, Sometimes, Often, |
| | Always |
| How often did your fatigue limit you at work | Never, Rarely, Sometimes, Often, |
| (include work at home)? | Always |
| How often were you too tired to think clearly? | Never, Rarely, Sometimes, Often, |
| | Always |
| How often were you too tired to take a bath or | Never, Rarely, Sometimes, Often, |
| shower? | Always |
| How often did you have enough energy to | Never, Rarely, Sometimes, Often, |
| exercise strenuously? | Always |

TABLE 4
CES-D (rarely or none of the time (less than 1 day), Some or
a little of the time (1-2 days), Occasionally or a moderate
amount of time (3-4 days), Most or all of the time (5-7 days)

| Item No. | |
|---|---|
| 1 | I was bothered by things that usually don't bother me. |
| 2 | I did not feel like eating; my appetite was poor. |
| 3 | I felt that I could not shake off the blues even with help from my family or |
| | friends. |
| 4 | I felt I was just as good as other people. |
| 5 | I had trouble keeping my mind on what I was doing. |
| 6 | I felt depressed. |
| 7 | I felt that everything I did was an effort. |
| 8 | I felt hopeful about the future. |
| 9 | I thought my life had been a failure. |
| 10 | I felt fearful. |
| 11 | My sleep was restless. |
| 12 | I was happy. |
| 13 | I talked less than usual. |
| 14 | I felt lonely. |
| 15 | People were unfriendly. |
| 16 | I enjoyed life. |
| 17 | I had crying spells. |
| 18 | I felt sad. |
| 19 | I felt that people dislike me. |
| 20 | I could not get “going.” |

TABLE 5
Parental Stress Scale Items

| Item | |
|---|---|
| No. | |
| 1 | The presence of monitors and equipment |
| 2 | The constant noise of monitors and alarms |
| 3 | The sudden noise of monitors and alarms |
| 4 | The other sick babies in the room |
| 5 | The large number of people working in the unit |
| 6 | Having a machine breathe for my baby |
| 7 | Tubes and equipment on or near my baby |
| 8 | Bruises, cuts or incisions on my baby |
| 9 | The unusual color of my baby |
| 10 | My baby's unusual or abnormal breathing pattern |
| 11 | The small size of my baby |
| 12 | The wrinkled appearance of my baby |
| 13 | Seeing needles and tubes put in my baby |
| 14 | My baby being fed by an intravenous drip |
| 15 | When my baby looked to be in pain |
| 16 | When my baby looked sad |
| 17 | The limp and weak appearance of my baby |
| 18 | Jerky or restless movements of my baby |
| 19 | My baby not being able to cry like other babies |
| 20 | Being separated from my baby |
| 21 | Not feeding my baby myself |
| 22 | Not being able to care for my baby myself (e.g. nappy changing) |
| 23 | Not being able to hold my baby when I want |
| 24 | Feeling helpless and unable to protect my baby from pain |
| 25 | Feeling helpless about how to help my baby during this time |
| 26 | Not being able to be alone with my baby |
| 27 | Staff explaining things too fast |
| 28 | Staff using words I don't understand |
| 29 | Telling me different (conflicting) things about my baby's condition |
| 30 | Not telling me enough about tests and |
| | treatments being done to my baby |
| 31 | Not talking to me enough |
| 32 | Too many different people talking to me |
| 33 | Difficulty in getting information or help when I visit or telephone |
| 34 | Not feeling sure that I will be called about changes in my baby's |
| | condition |
| 35 | Staff looking worried about my baby |
| 36 | Staff acting as if they didn't want parents around |
| 37 | Staff acting as if they did not understand |
| | my baby's behavior or special needs |

b. Mitigation of Pmads: Initial Feasibility Study

Initial pilot of COURAGE with 9 parents of infants hospitalized at the NICU of a hospital in Philadelphia.

A psychologist showed each parent 3 VR videos in a single session showcasing (a) NICU environment with alarm monitor, ventilator, and a nurse with a frail intubated mannequin baby, (b) Intubation process and (c) Resuscitation procedure with a neonatologist, respiratory specialist, nurse, and other staff in a tense environment. Before the session, psychometric measurements were taken including IES-R (Impact of Events Scale-Revised) for trauma/PTSD. After the session, usability scores were obtained, followed by a repeat of the psychometric measures one week after the session. There was a reduction in the IES-R measure (x̅ change=−7.44) depicting improvement in trauma symptoms and that the outcomes were trending in a positive direction. There were also positive results observed using Likert scales to assess key critical use parameters, 1 to 5 for low to high, such as likelihood to use (x̅=4.14), ease of use (x̅=4.77), comfort level with VR equipment (x̅=4.22), and ability to talk with the therapist while using the system (x̅=4.55). When asked how the NICU VR experience made them feel, parents trended towards less stressful responses (x̅=2.66; min 1-max 3).

c. Vr-Sit Study: Utilizing Virtual Reality Technology as a Stress Inoculation Tool for Nicu Caregivers

Annually, 1-7% of birthing parents delivering at full-term experience postpartum post-traumatic stress disorder (PTSD), while those giving birth to a pre-term or high-risk infant have a higher prevalence of PTSD, ranging from 24 to 44%. The prevalence of postpartum anxiety for full-term parents ranges from 13 to 40% but is higher for NICU caregivers, ranging from 24-35%, respectively. With the number of infants in the NICU increasing from 3.2 to 4.5 patients per 1000 live births between 2006 and 2010, and the associated prevalence of postpartum PTSD and anxiety in NICU caregivers, there is clearly a critical need to address parent mental health in the NICU setting. Decreasing the experience of stress for caregivers will allow for better mental health outcomes and thereby promote adaptive caregiver-infant interactions to support healthy child development.

To address PTSD, virtual reality (VR) has become increasingly more recognized in psychotherapy and psychological research with its ability to control the environment and create more stimulus and responsive protocols. VR integrates real-time computer graphics, body tracking devices, visual displays, and sensory outputs, putting a participant in computer-generated environments that can move in time with a person's head and body motion, giving more possibility for exposure therapy and mindfulness coaching. VR with supportive coaching has been shown to work effectively with veterans to decrease PTSD and PTSD symptoms.

Using VR scenarios and trained coaches with a four-session Stress Inoculation Training (SIT) protocol, it is aimed to decrease the prevalence of adverse stress responses in NICU caregivers. The goal of SIT is to teach skills to enhance resistance to stress and prepare individuals to respond to negative stress events. There are three phases to the SIT process: conceptualization, inoculation, and application. A meta-analysis of 37 studies showed that SIT was deemed an effective means for reducing state anxiety and enhancing performance under stress.

The purpose of this example is to provide SIT specifically designed for NICU parents using VR technology. The study uses a web-secure VR-enabled telemedicine platform (called COURAGE™) to administer VR-supported stress inoculation to NICU caregivers and assess their anxiety and PTSD symptoms using validated self-report measures. Evaluation of whether the delivery of VR-enhanced SIT can result in a demonstrable decrease in stress response and mental health symptoms for NICU parents is performed. An existing library of VR video scenarios (i.e., assets) are expanded (with input from previous NICU caregivers) and used by coaches to help current NICU caregivers (1) become proficient in relaxation skills and (2) apply these skills in experiential practice in order to reduce the stress response.

It is envisioned that the use of VR-supported coaching and stress inoculation will help decrease postpartum stress symptoms in NICU caregivers. For this study, the feasibility and efficacy of the COURAGE platform and SIT protocol for NICU caregivers are being tested. Overall, it is believed that teaching NICU caregivers how to properly assess and manage their stress will help lessen the experience of postpartum PTSD and anxiety symptoms. In future studies, we will examine whether SIT sessions with VR that are personalized to each caregiver will be more effective at decreasing PTSD and anxiety symptoms than non-VR-supported SIT sessions.

d. Coaching Pilot Study

This example is a coaching pilot that was performed completely remote with mothers across the U.S. in urban and rural areas. The results showed that there was a reduction in stress and anxiety levels by at least 20% pre to post session, 68% overall. The clients found the coaching very helpful in relieving stress and learning new techniques for mental wellness. The practitioners also found it very easy and useful to use with their patients (Table 6).

TABLE 6

| | | | |
|---|---|---|---|
| Results | Somewhat Helpful | Helpful | Very Helpful |

| | |
|---|---|
| Relieving Stress, accessing peace & | [symbol] |
| calm | |
| Learning new techniques | [symbol] |

| | |
|---|---|
| Better Sleep Quality | [symbol] |

e. Nicu Advocacy Group

In this example, a focus group was developed with family partners who graduated from the NICU. Participants were shown online videos (YouTube videos) and VR scenarios and were asked which type of format was more effective. The participants responded that the VR scenarios were more visceral and effective than the YouTube videos by far.

f. Testimonials

The effectiveness of the systems and methods are evidenced by the following testimonials provided by patients and practitioners.

### Patient Testimonials

“A few moments alone without distractions to just breathe”
“The longer guided meditation really helped me relax and do a full body scan” Practitioner Testimonials
“Love having the VR modality and platform as a tool to work with clients on together”
“We practice in real time and then they can do it on their own with confidence”

The foregoing descriptions of specific implementations have been presented for purposes of illustration and description. They are not intended to be exhaustive or to limit the disclosure to the precise forms disclosed, and modifications and variations are possible in view of the above teaching. The example implementations were chosen and described to best explain the principles of the disclosure and its practical application, to thereby enable others skilled in the art to best utilize the disclosure and its implementations with modifications as suited to the use contemplated.

It is therefore submitted that the invention has been shown and described in the most practical and exemplary implementations. It should be recognized that departures may be made which fall within the scope of the invention. With respect to the description provided herein, it is submitted that the optimal features of the invention include variations in size, materials, shape, form, function, manner of operation, assembly, and use. All structures, functions, and relationships equivalent or essentially equivalent to those disclosed are intended to be encompassed by the invention.

Any discussion of documents or subject matter included in the present disclosure is not to be taken as an admission that any or all of these materials form part of the prior art base or were common general knowledge in the field relevant to the present disclosure as it existed before the priority date of each claim of this and any continuing application.

While illustrative embodiments have been illustrated and described, it will be appreciated that various changes can be made therein without departing from the spirit and scope of the invention.

### NON-LIMITING EMBODIMENTS

While general features of the disclosure are described and shown and particular features of the disclosure are set forth in the claims, the following non-limiting embodiments relate to features, and combinations of features, that are explicitly envisioned as being part of the disclosure. The following non-limiting Embodiments contain elements that are modular and can be combined with each other in any number, order, or combination to form a new non-limiting Embodiment, which can itself be further combined with other non-limiting Embodiments.

Embodiment 1. A system for mental healthcare management for a patient in need thereof, the system comprising: circuitry configured to simultaneously administer: a virtual therapy session to a patient device; and a virtual therapy management session to a practitioner device; wherein the virtual therapy session and the virtual therapy management session are operably connected for exchange of data therebetween for an experience of virtual therapy by a patient and management of the experience by the patient, a practitioner, or both.

Embodiment 2. The system of any other Embodiment, further comprising: circuitry configured to: receive values of healthcare parameters of the patient; normalize the values of healthcare parameters; and quantify a health status of the patient based on the normalized values.

Embodiment 3. The system of any other Embodiment, wherein the health status of the patient is a numerical score that results from summation of the normalized values of the healthcare parameters.

Embodiment 4. The system of any other Embodiment, further comprising: circuitry configured to receive an input from the patient device in response to at least one survey for values of healthcare parameters and the health status; and circuitry configured to receive an input from the practitioner device for management of a care plan for the patient based at least in part on the health status.

Embodiment 5. The system of any other Embodiment, further comprising: circuitry configured for simultaneous transmission of media to both the patient device and the practitioner device; wherein the media is depicted to the patient and the practitioner via the patient device and the practitioner device, respectively, during the virtual therapy session and the virtual therapy management session, respectively.

Embodiment 6. The system of any other Embodiment, further comprising: circuitry configured to recommend media for the virtual therapy session and the virtual therapy management session based on a predicted responsiveness of the patient to the media, wherein the media for the virtual therapy session comprises a plurality of virtual videos that are combined together into a playlist for the patient.

Embodiment 7. The system of any other Embodiment, wherein the predicted responsiveness of the patient is based at least in part on a previous responsiveness of the patient to the media and/or another media, and/or a previous responsiveness of another patient to the media and/or another media.

Embodiment 8. A patient device configured for virtual therapy, the patient device comprising: circuitry configured to access a virtual therapy session of a system for mental healthcare management; and circuitry configured to depict media and practitioner input of the virtual therapy session to a patient.

Embodiment 9. The patient device of any other Embodiment, further comprising circuitry configured to receive a patient input from the patient and transmit the patient input to the system, whereby the patient input is accessible to a practitioner device in real-time with the virtual therapy session.

Embodiment 10. The patient device of any other Embodiment, wherein the practitioner device further comprises circuitry configured to receive a practitioner input from a practitioner and transmit the practitioner input to the system, whereby the practitioner input is accessible to the patient device in real-time with the virtual therapy session.

Embodiment 11. The patient device of any other Embodiment, wherein the patient device comprises at least one processor and at least one non-transitory machine-readable storage medium having stored thereon instructions which, when executed by the processor, configure the processor to: access the virtual therapy session of the system for mental healthcare management; and depict media and practitioner input of the virtual therapy session to the patient; and optionally: receive the patient input from the patient and transmit the patient input to the system.

Embodiment 12. The patient device of any other Embodiment, wherein the patient device is a smartphone, an augmented reality (AR) device, and/or a virtual reality (VR) device.

Embodiment 13. A practitioner device configured for virtual therapy management, the practitioner device comprising: circuitry configured to access a virtual therapy management session of a system for mental healthcare management; and circuitry configured to depict media and patient input of the virtual therapy management session to a practitioner.

Embodiment 14. The practitioner device of any other Embodiment, further comprising circuitry configured to receive a practitioner input from the practitioner and transmit the practitioner input to the system, whereby the practitioner input is accessible to a patient device in real-time with the virtual therapy management session.

Embodiment 15. The practitioner device of any other Embodiment, wherein the patient device further comprises circuitry configured to receive a patient input from a patient and transmit the patient input to the system, whereby the patient input is accessible to the practitioner device in real-time with the virtual therapy management session.

Embodiment 16. The practitioner device of any other Embodiment, wherein the practitioner device comprises at least one processor and at least one non-transitory machine-readable storage medium having stored thereon instructions which, when executed by the processor, configure the processor to: access the virtual therapy management session of the system for mental healthcare management; and depict media and patient input of the virtual therapy session to the practitioner; and optionally: receive the practitioner input from the practitioner and transmit the practitioner input to the system.

Embodiment 17. A method for mental healthcare management, the method comprising: depicting, to a patient and a practitioner, media for virtual therapy for the patient; receiving, from the patient, patient input related to an experience of the virtual therapy by the patient; and providing, by the practitioner, practitioner input related to the experience of the virtual therapy by the patient.

Embodiment 18. The method of any other Embodiment, wherein the virtual therapy comprises stress inoculation therapy and wherein the experience of the media by the patient is experiential or relaxational; wherein the practitioner input comprises selection of media for virtual therapy for the patient.

Embodiment 19. The method of any other Embodiment, wherein the virtual therapy comprises: virtual cognitive behavioral therapy (CBT) for inoculating the patient against stress and/or developing stress management techniques by the patient; virtual coaching and/or therapy for guiding and/or preparing the patient, wherein the coaching and/or therapy comprises psychoeducation tours or other media; and/or virtual coaching and/or therapy for preparing the patient for preconception, birthing, and/or postpartum.

Embodiment 20. The method of any other Embodiment, wherein the patient has or is at risk of having a perinatal mood and anxiety disorder (PMAD) and wherein the virtual therapy averts development or progression of the PMAD in the patient.

The embodiments of the invention in which an exclusive property or privilege is claimed are defined as follows:

1. A system for mental healthcare management for a patient in need thereof, the system comprising:
circuitry configured to simultaneously administer:
a virtual therapy session to a patient device; and
a virtual therapy management session to a practitioner device;
wherein the virtual therapy session and the virtual therapy management session are operably connected for exchange of data therebetween for an experience of virtual therapy by a patient and management of the experience by the patient, a practitioner, or both.

2. The system of claim 1, further comprising:
circuitry configured to:
receive values of healthcare parameters of the patient;
normalize the values of healthcare parameters; and
quantify a health status of the patient based on the normalized values.

3. The system of claim 2, wherein the health status of the patient is a numerical score that results from summation of the normalized values of the healthcare parameters.

4. The system of one of claims 2-3, further comprising:
circuitry configured to receive an input from the patient device in response to at least one survey for values of healthcare parameters and the health status; and
circuitry configured to receive an input from the practitioner device for management of a care plan for the patient based at least in part on the health status.

5. The system of one of claims 1-4, further comprising:
circuitry configured for simultaneous transmission of media to both the patient device and the practitioner device;
wherein the media is depicted to the patient and the practitioner via the patient device and the practitioner device, respectively, during the virtual therapy session and the virtual therapy management session, respectively.

6. The system of one of claims 1-5, further comprising:
circuitry configured to recommend media for the virtual therapy session and the virtual therapy management session based on a predicted responsiveness of the patient to the media, wherein the media for the virtual therapy session comprises a plurality of virtual videos that are combined together into a playlist for the patient.

7. The system of claim 6, wherein the predicted responsiveness of the patient is based at least in part on a previous responsiveness of the patient to the media and/or another media, and/or a previous responsiveness of another patient to the media and/or another media.

8. A patient device configured for virtual therapy, the patient device comprising:
circuitry configured to access a virtual therapy session of a system for mental healthcare management; and
circuitry configured to depict media and practitioner input of the virtual therapy session to a patient.

9. The patient device of claim 8, further comprising circuitry configured to receive a patient input from the patient and transmit the patient input to the system, whereby the patient input is accessible to a practitioner device in real-time with the virtual therapy session.

10. The patient device of claim 9, wherein the practitioner device further comprises circuitry configured to receive a practitioner input from a practitioner and transmit the practitioner input to the system, whereby the practitioner input is accessible to the patient device in real-time with the virtual therapy session.

11. The patient device of one of claims 8-10, wherein the patient device comprises at least one processor and at least one non-transitory machine-readable storage medium having stored thereon instructions which, when executed by the processor, configure the processor to:
access the virtual therapy session of the system for mental healthcare management; and
depict media and practitioner input of the virtual therapy session to the patient;
and optionally:
receive the patient input from the patient and transmit the patient input to the system.

12. The patient device of one of claims 8-10, wherein the patient device is a smartphone, an augmented reality (AR) device, and/or a virtual reality (VR) device.

13. A practitioner device configured for virtual therapy management, the practitioner device comprising:
circuitry configured to access a virtual therapy management session of a system for mental healthcare management; and
circuitry configured to depict media and patient input of the virtual therapy management session to a practitioner.

14. The practitioner device of claim 13, further comprising circuitry configured to receive a practitioner input from the practitioner and transmit the practitioner input to the system, whereby the practitioner input is accessible to a patient device in real-time with the virtual therapy management session.

15. The practitioner device of claim 14, wherein the patient device further comprises circuitry configured to receive a patient input from a patient and transmit the patient input to the system, whereby the patient input is accessible to the practitioner device in real-time with the virtual therapy management session.

16. The practitioner device of one of claims 13-15, wherein the practitioner device comprises at least one processor and at least one non-transitory machine-readable storage medium having stored thereon instructions which, when executed by the processor, configure the processor to:
access the virtual therapy management session of the system for mental healthcare management; and
depict media and patient input of the virtual therapy session to the practitioner;
and optionally:
receive the practitioner input from the practitioner and transmit the practitioner input to the system.

17. A method for mental healthcare management, the method comprising:
depicting, to a patient and a practitioner, media for virtual therapy for the patient;
receiving, from the patient, patient input related to an experience of the virtual therapy by the patient; and
providing, by the practitioner, practitioner input related to the experience of the virtual therapy by the patient.

18. The method of claim 17, wherein the virtual therapy comprises stress inoculation therapy and wherein the experience of the media by the patient is experiential or relaxational; wherein the practitioner input comprises selection of media for virtual therapy for the patient.

19. The method of one of claims 17-18, wherein the virtual therapy comprises:
virtual cognitive behavioral therapy (CBT) for inoculating the patient against stress and/or developing stress management techniques by the patient;
virtual coaching and/or therapy for guiding and/or preparing the patient, wherein the coaching and/or therapy comprises psychoeducation tours or other media; and/or
virtual coaching and/or therapy for preparing the patient for preconception, birthing, and/or postpartum.

20. The method of one of claims 17-19, wherein the patient has or is at risk of having a perinatal mood and anxiety disorder (PMAD) and wherein the virtual therapy averts development or progression of the PMAD in the patient.
